SEQUENCE LISTING (25 sequences)

SEQ ID NO: 1 - protein, 1970 aa, human
MHGGGPPSGDSACPLRTIKRVQFGVLSPDELKRMSVTEGGIKYPETTEGGRPKLGGLMDPRQGVIERTGRCQTCAGNMTECPGHFGHIELAKPVFHVGFLVKTMKVLRCVCFFCSKLLVDSNNPKIKDILAKSKGQPKKRLTHVYDLCKGKNICEGGEEMDNKFGVEQPEGDEDLTKEKGHGGCGRYQPRIRRSGLELYAEWKHVNEDSQEKKILLSPERVHEIFKRISDEECFVLGMEPRYARPEWMIVTVLPVPPLSVRPAVVMQGSARNQDDLTHKLADIVKINNQLRRNEQNGAAAHVIAEDVKLLQFHVATMVDNELPGLPRAMQKSGRPLKSLKQRLKGKEGRVRGNLMGKRVDFSARTVITPDPNLSIDQVGVPRSIAANMTFAEIVTPFNIDRLQELVRRGNSQYPGAKYIIRDNGDRIDLRFHPKPSDLHLQTGYKVERHMCDGDIVIFNRQPTLHKMSMMGHRVRILPWSTFRLNLSVTTPYNADFDGDEMNLHLPQSLETRAEIQELAMVPRMIVTPQSNRPVMGIVQDTLTAVRKFTKRDVFLERGEVMNLLMFLSTWDGKVPQPAILKPRPLWTGKQIFSLIIPGHINCIRTHSTHPDDEDSGPYKHISPGDTKVVVENGELIMGILCKKSLGTSAGSLVHISYLEMGHDITRLFYSNIQTVINNWLLIEGHTIGIGDSIADSKTYQDIQNTIKKAKQDVIEVIEKAHNNELEPTPGNTLRQTFENQVNRILNDARDKTGSSAQKSLSEYNNFKSMVVSGAKGSKINISQVIAVVGQQNVEGKRIPFGFKHRTLPHFIKDDYGPESRGFVENSYLAGLTPTEFFFHAMGGREGLIDTAVKTAETGYIQRRLIKSMESVMVKYDATVRNSINQVVQLRYGEDGLAGESVEFQNLATLKPSNKAFEKKFRFDYTNERALRRTLQEDLVKDVLSNAHIQNELEREFERMREDREVLRVIFPTGDSKVVLPCNLLRMIWNAQKIFHINPRLPSDLHPIKVVEGVKELSKKLVIVNGDDPLSRQAQENATLLFNIHLRSTLCSRRMAEEFRLSGEAFDWLLGEIESKFNQAIAHPGEMVGALAAQSLGEPATQMTLNTFHYAGVSAKNVTLGVPRLKELINISKKPKTPSLTVFLLGQSARDAERAKDILCRLEHTTLRKVTANTAIYYDPNPQSTVVAEDQEWVNVYYEMPDFDVARISPWLLRVELDRKHMTDRKLTMEQIAEKINAGFGDDLNCIFNDDNAEKLVLRIRIMNSDENKMQEEEEVVDKMDDDVFLRCIESNMLTDMTLQGIEQISKVYMHLPQTDNKKKIIITEDGEFKALQEWILETDGVSLMRVLSEKDVDPVRTTSNDIVEIFTVLGIEAVRKALERELYHVISFDGSYVNYRHLALLCDTMTCRGHLMAITRHGVNRQDTGPLMKCSFEETVDVLMEAAAHGESDPMKGVSENIMLGQLAPAGTGCFDLLLDAEKCKYGMEIPTNIPGLGAAGPTGMFFGSAPSPMGGISPAMTPWNQGATPAYGAWSPSVGSGMTPGAAGFSPSAASDASGFSPGYSPAWSPTPGSPGSPGPSSPYIPSPGGAMSPSYSPTSPAYEPRSPGGYTPQSPSYSPTSPSYSPTSPSYSPTSPNYSPTSPSYSPTSPSYSPTSPSYSPTSPSYSPTSPSYSPTSPSYSPTSPSYSPTSPSYSPTSPSYSPTSPSYSPTSPSYSPTSPSYSPTSPSYSPTSPSYSPTSPSYSPTSPNYSPTSPNYTPTSPSYSPTSPSYSPTSPNYTPTSPNYSPTSPSYSPTSPSYSPTSPSYSPSSPRYTPQSPTYTPSSPSYSPSSPSYSPTSPKYTPTSPSYSPSSPEYTPTSPKYSPTSPKYSPTSPKYSPTSPTYSPTTPKYSPTSPTYSPTSPVYTPTSPKYSPTSPTYSPTSPKYSPTSPTYSPTSPKGSTYSPTSPGYSPTSPTYSLTSPAISPDDSDEEN

SEQ ID NO: 2 - protein, 1733 aa, yeast
MVGQQYSSAPLRTVKEVQFGLFSPEEVRAISVAKIRFPETMDETQTRAKIGGLNDPRLGSIDRNLKCQTCQEGMNECPGHFGHIDLAKPVFHVGFIAKIKKVCECVCMHCGKLLLDEHNELMRQALAIKDSKKRFAAIWTLCKTKMVCETDVPSEDDPTQLVSRGGCGNTQPTIRKDGLKLVGSWKKDRATGDADEPELRVLSTEEILNIFKHISVKDFTSLGFNEVFSRPEWMILTCLPVPPPPVRPSISFNESQRGEDDLTFKLADILKANISLETLEHNGAPHHAIEEAESLLQFHVATYMDNDIAGQPQALQKSGRPVKSIRARLKGKEGRIRGNLMGKRVDFSARTVISGDPNLELDQVGVPKSIAKTLTYPEVVTPYNIDRLTQLVRNGPNEHPGAKYVIRDSGDRIDLRYSKRAGDIQLQYGWKVERHIMDNDPVLFNRQPSLHKMSMMAHRVKVIPYSTFRLNLSVTSPYNADFDGDEMNLHVPQSEETRAELSQLCAVPLQIVSPQSNKPCMGIVQDTLCGIRKLTLRDTFIELDQVLNMLYWVPDWDGVIPTPAIIKPKPLWSGKQILSVAIPNGIHLQRFDEGTTLLSPKDNGMLIIDGQIIFGVVEKKTVGSSNGGLIHVVTREKGPQVCAKLFGNIQKVVNFWLLHNGFSTGIGDTIADGPTMREITETIAEAKKKVLDVTKEAQANLLTAKHGMTLRESFEDNVVRFLNEARDKAGRLAEVNLKDLNNVKQMVMAGSKGSFINIAQMSACVGQQSVEGKRIAFGFVDRTLPHFSKDDYSPESKGFVENSYLRGLTPQEFFFHAMGGREGLIDTAVKTAETGYIQRRLVKALEDIMVHYDNTTRNSLGNVIQFIYGEDGMDAAHIEKQSLDTIGGSDAAFEKRYRVDLLNTDHTLDPSLLESGSEILGDLKLQVLLDEEYKQLVKDRKFLREVFVDGEANWPLPVNIRRIIQNAQQTFHIDHTKPSDLTIKDIVLGVKDLQENLLVLRGKNEIIQNAQRDAVTLFCCLLRSRLATRRVLQEYRLTKQAFDWVLSNIEAQFLRSVVHPGEMVGVLAAQSIGEPATQMTLNTFHFAGVASKKVTSGVPRLKEILNVAKNMKTPSLTVYLEPGHAADQEQAKLIRSAIEHTTLKSVTIASEIYYDPDPRSTVIPEDEEIIQLHFSLLDEEAEQSFDQQSPWLLRLELDRAAMNDKDLTMGQVGERIKQTFKNDLFVIWSEDNDEKLIIRCRVVRPKSLDAETEAEEDHMLKKIENTMLENITLRGVENIERVVMMKYDRKVPSPTGEYVKEPEWVLETDGVNLSEVMTVPGIDPTRIYTNSFIDIMEVLGIEAGRAALYKEVYNVIASDGSYVNYRHMALLVDVMTTQGGLTSVTRHGFNRSNTGALMRCSFEETVEILFEAGASAELDDCRGVSENVILGQMAPIGTGAFDVMIDEESLVKYMPEQKITEIEDGQDGGVTPYSNESGLVNADLDVKDELMFSPLVDSGSNDAMAGGFTAYGGADYGEATSPFGAYGEAPTSPGFGVSSPGFSPTSPTYSPTSPAYSPTSPSYSPTSPSYSPTSPSYSPTSPSYSPTSPSYSPTSPSYSPTSPSYSPTSPSYSPTSPSYSPTSPSYSPTSPSYSPTSPSYSPTSPSYSPTSPSYSPTSPAYSPTSPSYSPTSPSYSPTSPSYSPTSPSYSPTSPNYSPTSPSYSPTSPGYSPGSPAYSPKQDEQKHNENENSR

SEQ ID NO: 3 - protein, 1407 aa, E. coli [sequence omitted]

SEQ ID NO: 4 - protein, 1174 aa, human
MYDADEDMQYDEDDDEITPDLWQEACWIVISSYFDEKGLVRQQLDSFDEFIQMSVQRIVEDAPPIDLQAEAQHASGEVEEPPRYLLKFEQIYLSKPTHWERDGAPSPMMPNEARLRNLTYSAPLYVDITKTVIKEGEEQLQTQHQKTFIGKIPIMLRSTYCLLNGLTDRDLCELNECPLDPGGYFIINGSEKVLIAQEKMATNTVYVFAKKDSKYAYTGECRSCLENSSRPTSTIWVSMLARGGQGAKKSAIGQRIVATLPYIKQEVPIIIVFRALGFVSDRDILEHIIYDFEDPEMMEMVKPSLDEAFVIQEQNVALNFIGSRGAKPGVTKEKRIKYAKEVLQKEMLPHVGVSDFCETKKAYFLGYMVHRLLLAALGRRELDDRDHYGNKRLDLAGPLLAFLFRGMFKNLLKEVRIYAQKFIDRGKDFNLELAIKTRIISDGLKYSLATGNWGDQKKAHQARAGVSQVLNRLTFASTLSHLRRLNSPIGRDGKLAKPRQLHNTLWGMVCPAETPEGHAVGLVKNLALMAYISVGSQPSPILEFLEEWSMENLEEISPAAIADATKIFVNGCWVGIHKDPEQLMNTLRKLRRQMDIIVSEVSMIRDIREREIRIYTDAGRICRPLLIVEKQKLLLKKRHIDQLKEREYNNYSWQDLVASGVVEYIDTLEEETVMLAMTPDDLQEKEVAYCSTYTHCEIHPSMILGVCASIIPFPDHNQSPRNTYQSAMGKQAMGVYITNFHVRMDTLAHVLYYPQKPLVTTRSMEYLRFRELPAGINSIVAIASYTGYNQEDSVIMNRSAVDRGFFRSVFYRSYKEQESKKGFDQEEVFEKPTRETCQGMRHAIYDKLDDDGLIAPGVRVSGDDVIIGKTVTLPENEDELESTNRRYTKRDCSTFLRTSETGIVDQVMVTLNQEGYKFCKIRVRSVRIPQIGDKFASRHGQKGTCGIQYRQEDMPFTCEGITPDIIINPHAIPSRMTIGHLIECLQGKVSANKGEIGDATPFNDAVNVQKISNLLSDYGYHLRGNEVLYNGFTGRKITSQIFIGPTYYQRLKHMVDDKIHSRARGPIQILNRQPMEGRSRDGGLRFGEMERDCQIAHGAAQFLRERLFEASDPYQVHVCNLCGIMAIANTRTHTYECRGCRNKTQISLVRMPYACKLLFQELMSMSIAPRMMSV

SEQ ID NO: 5 - protein, 1224 aa, yeast
MSDLANSEKYYDEDPYGFEDESAPITAEDSWAVISAFFREKGLVSQQLDSFNQFVDYTLQDIICEDSTLILEQLAQHTTESDNISRKYEISFGKIYVTKPMVNESDGVTHALYPQEARLRNLTYSSGLFVDVKKRTYEAIDVPGRELKYELIAEESEDDSESGKVFIGRLPIMLRSKNCYLSEATESDLYKLKECPFDMGGYFIINGSEKVLIAQERSAGNIVQVFKKAAPSPISHVAEIRSALEKGSRFISTLQVKLYGREGSSARTIKATLPYIKQDIPIVIIFRALGIIPDGEILEHICYDVNDWQMLEMLKPCVEDGFVIQDRETALDFIGRRGTALGIKKEKRIQYAKDILQKEFLPHITQLEGFESRKAFFLGYMINRLLLCALDRKDQDDRDHFGKKRLDLAGPLLAQLFKTLFKKLTKDIFRYMQRTVEEAHDFNMKLAINAKTITSGLKYALATGNWGEQKKAMSSRAGVSQVLNRYTYSSTLSHLRRTNTPIGRDGKLAKPRQLHNTHWGLVCPAETPEGQACGLVKNLSLMSCISVGTDPMPIITFLSEWGMEPLEDYVPHQSPDATRVFVNGVWHGVHRNPARLMETLRTLRRKGDINPEVSMIRDIREKELKIFTDAGRVYRPLFIVEDDESLGHKELKVRKGHIAKLMATEYQDIEGGFEDVEEYTWSSLLNEGLVEYIDAEEEESILIAMQPEDLEPAEANEENDLDVDPAKRIRVSHHATTFTHCEIHPSMILGVAASIIPFPDHNQSPRNTYQSAMGKQAMGVFLTNYNVRMDTMANILYYPQKPLGTTRAMEYLKFRELPAGQNAIVAIACYSGYNQEDSMIMNQSSIDRGLFRSLFFRSYMDQEKKYGMSITETFEKPQRTNTLRMKHGTYDKLDDDGLIAPGVRVSGEDVIIGKTTPISPDEEELGQRTAYHSKRDASTPLRSTENGIVDQVLVTTNQDGLKFVKVRVRTTKIPQIGDKFASRHGQKGTIGITYRREDMPFTAEGIVPDLIINPHAIPSRMTVAHLIECLLSKVAALSGNEGDASPFTDITVEGISKLLREHGYQSRGFEVMYNGHTGKKLMAQIFFGPTYYQRLRHMVDDKIHARARGPMQVLTRQPVEGRSRDGGLRFGEMERDCMIAHGAASFLKERLMEASDAFRVHICGICGLMTVIAKLNHNQFECKGCDNKIDIYQIHIPYAAKLLFQELMAMNITPRLYTDRSRDF

SEQ ID NO: 6 - protein, 1342 aa, E. coli
  VARIANT 72, 516: Xaa = Any Amino Acid
MVYSYTEKKRIRKDFGKRPQVLDVPYLLSIQLDSFQKFIEQDPEGQYGLEAAFRSVFPIQSYSGNSELQYVXYRLGEPVFDVQECQIRGVTYSAPLRVKLRLVIYEREAPEGTVKDIKEQEVYMGEIPLMTDNGTFVINGTERVIVSQLHRSPGVFFDSDKGKTHSSGKVLYNARIIPYRGSWLDFEFDPKDNLFVRIDRRRKLPATIILRALNYTTEQILDLFFEKVIFEIRDNKLQMELVPERLRGETASFDIEANGKVYVEKGRRITARHIRQLEKDDVKLIEVPVEYIAGKVVAKDYIDESTGELICAANMELSLDLLAKLSQSGHKRIETLFTNDLDHGPYISETLRVDPTNDRLSALVEIYRMMRPGEPPTREAAESLFENLFFSEDRYDLSAVGRMKFNRSLLREEIEGSGILSKDDIIDVMKKLIDIRNGKGEVDDIDHLGNRRIRSVGEMAENQFRVGLVRVERAVKERLSLGDLDTLMPQDMINAKPISAAVKEFFGSSQLSQFMXQNNPLSEITHKRRISALGPGGLTRERAGFEVRDVHPTHYGRVCPIETPEGPNIGLINSLSVYAQTNEYGFLETPYRKVTDGVVTDEIHYLSAIEEGNYVIAQANSNLDEEGHFVEDLVTCRSKGESSLFSRDQVDYMDVSTQQVVSVGASLIPFLEHDDANRALMGANMQRQAVPTLRADKPLVGTGMERAVAVDSGVTAVAKRGGVVQYVDASRIVIKVNEDEMYPGEAGIDIYNLTKYTRSNQNTCINQMPCVSLGEPVERGDVLADGPSTDLGELALGQNMRVAFMPWNGYNFEDSILVSERVVQEDRFTTIHIQELACVSRDTKLGPEEITADIPNVGEAALSKLDESGIVYIGAEVTGGDILVGKVTPKGETQLTPEEKLLRAIFGEKASDVKDSSLRVPNGVSGTVIDVQVFTRDGVEKDKRALEIEEMQLKQAKKDLSEELQILEAGLFSRIRAVLVAGGVEAEKLDKLPRDRWLELGLTDEEKQNQLEQLAEQYDELKHEFEKKLEAKRRKITQGDDLAPGVLKIVKVYLAVKRRIQPGDKMAGRHGNKGVISKINPIEDMPYDENGTPVDIVLNPLGVPSRMNIGQILETHLGMAAKGIGDKINAMLKQQQEVAKLREFIQRAYDLGADVRQKVDLSTFSDEEVMRLAENLRKGMPIATPVFDGAKEAEIKELLKLGDLPTSGQIRLYDGRTGEQFERPVTVGYMYMLKLNHLVDDKMHARSTGSYSLVTQQPLGGKAQFGGQRFGEMEVWALEAYGAAYTLQEMLTVKSDDVNGRTKMYKNIVDGNHQMEPGMPESFNVLLKEIRSLGINIELEDE

SEQ ID NO: 7 - protein, 318 aa, yeast
MSEEGPQVKIREASKDNVDFILSNVDLAMANSLRRVMIAEIPTLAIDSVEVETNTTVLADEFIAHRLGLIPLQSMDIEQLEYSRDCFCEDHCDKCSVVLTLQAFGESESTTNVYSKDLVIVSNLMGRNIGHPIIQDKEGNGVLICKLRKGQELKLTCVAKKGIAKEHAKWGPAAAIEFEYDPWNKLKHTDYWYEQDSAKEWPQSKNCEYEDPPNEGDPFDYKAQADTFYMNVESVGSIPVDQVVVRGIDTLQKKVASILLALTQMDQDKVNFASGDNNTASNMLGSNEDVMMTGAEQDPYSNASQMGNTGSGGYDNAW

SEQ ID NO: 8 - protein, 275 aa, human
MPYANQPTVRITELTDENVKFIIENTDLAVANSIRRVFIAEVPIIAIDWVQIDANSSVLHDEFIAHRLGLIPLISDDIVDKLQYSRDCTCEEFCPECSVEFTLDVRCNEDQTRHVTSRDLISNSPRVIPVTSRNRDNDPNDYVEQDDILIVKLRKGQELRLRAYAKKGFGKEHAKWNPTAGVAFEYDPDNALRHTVYPKPEEWPKSEYSELDEDESQAPYDPNGKPERFYYNVESCGSLRPETIVLSALSGLKKKLSDLQTQLSHEIQSDVLTIN

SEQ ID NO: 9 - protein, 120 aa, yeast
MNAPDRFELFLLGEGESKLKIDPDTKAPNAVVITFEKEDHTLGNLIRAELLNDRKVLFAAYKVEHPFFARFKLRIQTTEGYDPKDALKNACNSIINKLGALKTNFETEWNLQTLAADDAF

SEQ ID NO: 10 - protein, 117 aa, human
MNAPPAFESFLLFEGEKKITINKDTKVPNACLFTINKEDHTLGNIIKSQLLKDPQVLFAGYKVPHPLEHKIIIRVQTTPDYSPQEAFTNAITDLISELSLLEERFRVAIKDKQEGIE

SEQ ID NO: 11 - protein, 70 aa, yeast
MIVPVRCFSCGKVVGDKWESYLNLLQEDELDEGTALSRLGLKRYCCRRMILTHVDLIEKFLRYNPLEKRD

SEQ ID NO: 12 - protein, 67 aa, human
MIIPVRCFTCGKIVGNKWEAYLGLLQAEYTEGDALDALGLKRYCCRRMLLAHVDLIEKLLNYAPLEK

SEQ ID NO: 13 - protein, 70 aa, yeast
MSREGFQIPTNLDAAAAGTSQARTATLKYICAECSSKLSLSRTDAVRCKDCGHRILLKARTKRLVQFEAR

SEQ ID NO: 14 - protein, 58 aa, human
MDTQKDVQPPKQQPMIYICGECHTENEIKSRDPIRCRECGYRIMYKKRTKRLVVFDAR

SEQ ID NO: 15 - protein, 215 aa, yeast
MDQENERNISRLWRAFRTVKEMVKDRGYFITQEEVELPLEDFKAKYCDSMGRPQRKMMSFQANPTEESISKFPDMGSLWVEFCDEPSVGVKTMKTFVIHIQEKNFQTGIFVYQNNITPSAMKLVPSIPPATIETFNEAALVVNITHHELVPKHIRLSSDEKRELLKRYRLKESQLPRIQRADPVALYLGLKRGEVVKIIRKSETSGRYASYRICM

SEQ ID NO: 16 - protein, 155 aa, yeast
MSDYEEAFNDGNENFEDFDVEHFSDEETYEEKPQFKDGETTDANGKTIVTGGNGPEDFQQHEQIRRKTLKEKAIPKDQRATTPYMTKYERARILGTRALQISMNAPVFVDLEGETDPLRIAMKELAEKKIPLVIRRYLPDGSFEDWSVEELIVDL

SEQ ID NO: 17 - protein, 146 aa, yeast
MSNTLFDDIFQVSEVDPGRYNKVCRIEAASTTQDQCKLTLDINVELFPVAAQDSLTVTIASSLNLEDTPANDSSATRSWRPPQAGDRSLADDYDYVMYGTAYKFEEVSKDLIAVYYSFGGLLMRLEGNYRNLNNLKQENAYLLIRR

SEQ ID NO: 18 - protein, 122 aa, yeast
MTTFRFCRDCNNMLYPREDKENNRLLFECRTCSYVEEAGSPLVYRHELITNIGETAGVVQDIGSDPTLPRSDRECPKCHSRENVFFQSQQRRKDTSMVLFFVCLSCSHIFTSDQKNKRTQFS

SEQ ID NO: 19 - protein, 210 aa, human
MDDEEETYRLWKIRKTIMQLCHDRGYLVTQDELDQTLEEFKAQFGDKPSEGRPRRTDLTVLVAHNDDPTDQMFVFFPEEPKVGIKTIKVYCQRMQEENITRALIVVQQGMTPSAKQSLVDMAPKYILEQFLEQELLINITEHELVPEHVVMTKEEVSELLARYKLRENQLPRIQAGDPVARYFGIRRGQVVKIIRPSETAGRYITYRLVQ

SEQ ID NO: 20 - protein, 127 aa, human
MSDNEDNFDGDDFDDVEEDEGLDDLENAEEEGQENVEILPSGERPQANQKRITTPYMTKYERARVLGTRALQIAMCAPVMVELEGETDPLLIAMKELKARKIPIIIRRYLPDGSYEDWGVDELIITD

SEQ ID NO: 21 - protein, 150 aa, human
MAGILFEDIFDVKDIDPEGKKFDRVSRLHCESESFKMDLILDVNIQIYPVDLGDKFRLVIASTLYEDGTLDDGEYNPTDDRPSRADQFEYVMYGKVYRIEGDETSTEAATRLSAYVSYGGLLMRLQGDANNLHGFEVDSRVYLLMKKLAF

SEQ ID NO: 22 - protein, 125 aa, human
MEPDGTYEPGFVGIRFCQECNNMLYPKEDKENRILLYACRNCDYQQEADNSCIYVNKITHEVDELTQIIADVSQDPTLPRTEDHPCQKCGHKEAVFFQSHSARAEDAMRLYYVCTAPHCGHRWTE

SEQ ID NO: 23 - DNA, 42 bp, human
cttccgcaacaagaaaaaatgcctggtcttcccccccccccc

SEQ ID NO: 24 - DNA, 33 bp, human
ggggaaggcgttgttcttttttacggacaagaa

SEQ ID NO: 25 - RNA, 14 nt, human
acggaccagaaggg

(19) United States
(12) Patent Application Publication
(10) Pub. No.: US 2008/0195324 A1
(43) Pub. Date: Aug. 14, 2008

(54) Computer comprising three-dimensional coordinates of a yeast RNA polymerase II

(76) Inventors: David A. Bushnell, Menlo Park, CA (US); Roger D. Kornberg, Atherton, CA (US); Patrick Cramer, Munich (DE)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/999,178

(22) Filed: Dec. 3, 2007

Related U.S. Application Data
(62) Division of application No. 10/418,772, filed on Apr. 17, 2003, now abandoned.
(60) Provisional application No. 60/373,486, filed on Apr. 17, 2002.

(51) Int. Cl. G06G 7/60; G01N 33/48

(52) U.S. Cl. 702/19; 703/11

(57) ABSTRACT

Crystals and structures are provided for an eukaryotic RNA polymerase, and an elongation complex containing a eukaryotic RNA polymerase. The structures and structural coordinates are useful in structural homology deduction, in developing and screening agents that affect the activity of eukaryotic RNA polymerase, and in designing modified forms of eukaryotic RNA polymerase. The structure information may be provided in a computer readable form, e.g. as a database of atomic coordinates, or as a three-dimensional model. The structures are useful, for example, in modeling interactions of the enzyme with DNA, RNA, transcription factors, nucleotides, etc. The structures are also used to identify molecules that bind to or otherwise interact with structural elements in the polymerase.

### BACKGROUND OF THE INVENTION

The control of gene transcription is essential to the functioning of cellular organisms. By regulating which genes are transcribed and when, the cell is able to respond to stimuli, proliferate, and differentiate. And when gene regulation goes awry, the consequences to the cell, and potentially to the organism, can be fatal.

The multisubunit enzyme RNA polymerase II (also called RNA polymerase b, Rpb, or Pol II) is the central enzyme of gene expression in eukaryotes. It reads the sequence of one strand of the DNA double helix (the template) and in so doing synthesizes messenger RNA (mRNA), which is then translated into protein. Pol II transcription is the first step in gene expression and a focal point of cell regulation. It is a target of many signal transduction pathways, and a molecular switch for cell differentiation in development.

Pol II stands at the center of complex machinery, whose composition changes in the course of gene transcription. This eukaryotic RNA polymerase comprises upwards of a dozen subunits with a total molecular mass of around 500 kDa. As many as six general transcription factors assemble with Pol II for promoter recognition and melting. A multiprotein Mediator transduces regulatory information from activators and repressors. Additional regulatory proteins interact with Pol II during RNA chain elongation, as do enzymes for RNA capping, splicing, and cleavage/polyadenylation.

Pol II is comprised of 12 subunits, with a total mass of greater than 0.5 MD. A backbone model of a 10-subunit yeast Pol II (lacking two small subunits dispensable for transcription) was previously obtained by x-ray diffraction and phase determination to approximately 3.5 Å resolution (Cramer et al. (2000) Science 288:640). The model revealed the general architecture of the enzyme and led to proposals for interactions with DNA and RNA in a transcribing complex.

RNA polymerase II (pol II) has been isolated in two forms, a 12-subunit “complete” enzyme and a 10-subunit “core.” The two additional subunits of the complete enzyme, Rpb4 and Rpb7, form a heterodimer and associate reversibly with core. The two enzymes are equivalent in RNA chain elongation, but core pol II is defective in the initiation of transcription. Addition of Rpb4/Rpb7 to core pol II restores initiation activity. Rpb4/Rpb7 may therefore be regarded as a general transcription factor, akin to the previously described TFIIB, -D, -E, -F, and -H.

Deletion of the RPB4 gene in yeast results in a temperature-sensitive phenotype, with cessation of growth above 32° C., while deletion of RPB7 is lethal. Microarray analysis reveals the rapid shutdown of 98% of all yeast mRNA synthesis upon shift of a Δrpb4 strain to a restrictive temperature, consistent with Rpb4/Rpb7 serving as a general transcription factor. Even at a permissive temperature, where constitutive gene transcription is not much affected by RPB4 deletion, transcription of inducible promoters is largely abolished. Overexpression of RPB7 suppresses many of the phenotypes of a Δrpb4 strain, but it fails to suppress the activation defect at most promoters tested. These results confirm the interaction of Rpb4 and Rpb7 in vivo, and show that the heterodimer also fits the definition of a transcriptional “coactivator.”

The incredible importance of RNA polymerase in cellular physiology makes its structural determination of great interest for development of therapeutic agents, for molecular design, and for manipulation of gene expression.

### Relevant Literature

Cramer et al. (2000) Science 288(5466):640-9 disclose the architecture of RNA polymerase II, and a backbone structure. Poglitsch et al., (1999) Cell 98(6):791-8 provide an electron crystal structure of an RNA polymerase II transcription elongation complex. Asturias et al. (1997) J Mol Biol. 272(4):536-40 reveal two conformations of RNA polymerase II by electron crystallography. Jensen et al., (1998) EMBO J. 17(8):2353-8 disclose the structure of wild-type yeast RNA polymerase II and location of Rpb4 and Rpb7. Fu et al., (1998) J Mol Biol. 280(3):317-22 disclose repeated tertiary fold of RNA polymerase II and implications for DNA binding. Gnatt et al., (1997) J Biol Chem. 272(49):30799-805 disclose the formation and crystallization of yeast RNA polymerase II elongation complexes. Fu et al. (1999) Cell 98(6):799-810 provide a structure of yeast RNA polymerase II at 5 A resolution.

A review of RNA polymerase II transcription factors may be found in Reinberg et al. (1998) Cold Spring Harb Symp Quant Biol. 63:83-103. Woychik (1998) Cold Spring Harb Symp Quant Biol. 63:311-7 reviews the function of RNA polymerase II. The mechanism and regulation of yeast RNA polymerase II transcription is discussed by Sayre and Kornberg (1993) Cell Mol Biol Res. 39(4):349-54.

U.S. Pat. No. 6,225,076, Darst et al., discloses a structure of a prokaryotic RNA polymerase.

### SUMMARY OF THE INVENTION

Methods and compositions are provided for modeling the structure of RNA polymerase II, and for identifying molecules that will bind to, and otherwise interact, with functional elements of the polymerase, thereby affecting transcription. The methods of the invention entail structural modeling, and the identification and design of molecules having a particular structure. The structural data obtained for the two forms of RNA polymerase II, for an elongation complex, for a complex with bound inhibitor, and for the complete 12 subunit enzyme can be used for the rational design of drugs that affect cell proliferation, gene expression, transcriptional fidelity, specificity of antibiotics, and the like.

The methods rely on the use of precise structural information derived from crystal structure studies of the RNA polymerase II. This structural data permits the identification of atoms that are important for a number of important structural elements. The enzyme has a complex structure, with a number of distinct elements that allow for the entry of a DNA double helix into the enzyme, the opening of the double helix and catalysis of synthesis of RNA on the DNA template, and the movement of DNA-RNA hybrid through the enzyme.

Such elements include the active site, and the position of metal ions within the active site. Atoms and coordinates are identified for the site for the entry of DNA into the enzyme and the clamp region, which includes a set of protein loops at the base of the clamp that act as pivots for DNA movement. The situation of the DNA double helix in the cleft formed between Rpb1 and Rpb2 are identified. A protein wall element is disclosed, which acts to block the straight passage of DNA into the enzyme, thereby forcing a bend in the DNA-RNA hybrid that exposes the end for addition of NTPs. A funnel shaped opening and pore to the active site are disclosed for the entry of NTPs. A loop of protein termed the rudder is identified, which abuts the 5′ end of the RNA and prevents extension of the DNA-RNA hybrid beyond 9 base pairs, separating DNA from RNA. The exit path of the RNA is identified as it passes beneath the rudder and beneath another loop of protein termed the lid, where the rudder and lid emanate from a massive clamp that swings over the active center region. A protein helix termed the bridge, which spans the cleft between Rpb1 and Rpb2, is disclosed as making hydrophobic contact with the base of the coding nucleotide in the template strand at the active site. The reversibly associated heterodimer of Rpb7 and Rpb4 is shown have contacts above the groove and the groove, bracketing the clamp, and constraining it in the closed state. The heterodimer may also interact with TFIIb to stabilize the transcription initiation complex, and with Mediator.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. Refined Pol II structure. (A) σ_{A}-weighted 2 mF_{obs}-DF_{calc} electron density at 2.8 Å resolution (green) superimposed on the final structure in crystal form 2. Three areas of the structure are shown: the packing of α helices in the foot region of Rpb1, a β strand in Rpb11, and the active-site loop in Rpb1. Backbone carbonyl oxygens are revealed in the map. An anomalous difference Fourier of the Mn²⁺-soaked crystal reveals the location of the active-site metal A (magenta, contoured at 10σ). An anomalous difference Fourier of a crystal of partially selenomethionine-substituted polymerase reveals the location of the S atom in residue M487 (white, contoured at 2.5σ). This figure was prepared with O. (B) Stereoview of a ribbon representation of the Pol II structure in form 2. Secondary structure was assigned by inspection. The diagram in the upper right corner is a key to the color code and an interaction diagram for the 10 subunits. The thickness of the connecting lines corresponds to the surface area buried in the corresponding subunit interface. This figure and others were prepared with RIBBONS.

FIG. 2. Structure of Rpb1. (A) Domains and domainlike regions of Rpb1. The amino acid residue numbers at the domain boundaries are indicated. (B) Ribbon diagrams, showing the location of Rpb1 within Pol II (“front” and “top” views of the enzyme), and Rpb1 alone. Locations of NH₂- and COOH-termini are indicated. Color-coding as in (A). (C) Secondary structure and amino acid sequence alignment. Yeast amino acid residue numbers are indicated above the sequence. Secondary structure elements were identified by inspection and are indicated and numbered above the sequence (boxes for α helices, arrows for β strands). Solid, dotted, and dashed lines above the sequences indicate ordered, partially ordered, and disordered loops, respectively. Alignment of Rpb1 from yeast (y) (SEQ ID NO:1) with human Rpb1 (h) (SEQ ID NO:2) and E. coli subunit β (e) (SEQ ID NO:3) was initially carried out with CLUSTALW and then edited by hand. Alignment of the E. coli sequence is based on the structure of the bacterial enzyme. Regions for which the polypeptide backbones follow the same course are indicated by gray bars below the sequences (dotted when uncertain). The remaining regions could not be aligned because of disorder or because they differ in structure so that alignment is meaningless. Sequence homology blocks A to H are indicated below the sequences by black bars. Important structural elements and prominent regions involved in subunit interactions are also noted. Residues involved in Zn²⁺ and Mg²⁺ coordination are highlighted in blue and pink, respectively. (D) Views of the domains and domainlike regions of Rpb1 (stereo on the left, mono on the right). These views reveal the entire course of the polypeptide chain from NH₂- to COOH-terminus and the locations of all secondary structure elements.

FIG. 3. (A to D) Structure of Rpb2. Organization and notation as in FIG. 2, except that the sequence alignment in (C) (SEQ ID NO:4), (SEQ ID NO:5) is with E. coli subunit D and its homology blocks A to I (SEQ ID NO:6).

FIG. 4. Structure and location of the Rpb3/10/11/12 subassembly. (A) Domain structure and sequence alignments. Rpb3 and Rpb11 from yeast (y3, y11) and human (h3, h11) were aligned with E. coli subunit α (eα) on the basis of comparison with the bacterial structure. Regions for which the polypeptide backbones follow the same course are indicated by gray bars. Rpb10 and Rpb12 from yeast (y) were aligned with the human subunits (h). See FIG. 2 for details. (B) Location of the Rpb3/10/11/12 subassembly in Pol II “back” view, of the enzyme. (C) Stereoview of the subassembly from the same direction as in (B).

FIG. 5. Structure and location of Rpb5, Rpb6, Rpb8, and Rpb9. (A) Domain structure and sequence alignments. The amino acid sequences of the yeast subunits (y) were aligned with those of the human subunits (h). Subunit Rpb6 was aligned with E. coli subunit ω (e). See FIG. 2 legend for details. (B) Location of the subunits in Pol II “side” view of the enzyme. (C) Stereoview of the subunits from the same direction as in (B), except for Rpb9, which is rotated 180° about a vertical axis.

FIG. 6. Surface charge distribution and factor binding sites. The surface of Pol II is colored according to the electrostatic surface potential, with negative, neutral, and positive charges shown in red, white, and blue, respectively. The active site is marked by a pink sphere. The asterisk indicates the location of the conserved start of a fragment of E. coli RNA polymerase subunit β that has been cross-linked to an extruded RNA 3′ end.

FIG. 7. Four mobile modules of the Pol II structure. (A) Backbone traces of the core, jaw-lobe, clamp, and shelf modules of the form 1 structure, shown in gray, blue, yellow, and pink, respectively. (B) Changes in the position of the jaw-lobe, clamp, and shelf modules between form 1 (colored) and form 2 structures (gray). The arrows indicate the direction of charges from form 1 to form 2. The core modules in the two crystal forms were superimposed and then omitted for clarity. (C) The view in (B) rotated 90° about a vertical axis. The core and jaw-lobe modules are omitted for clarity. In form 2, the clamp has swung to the left, opening a wider gap between its edge and the wall located further to the right.

FIG. 8. Active center. Stereoview from the Rpb2 side toward the clamp. Two metal ions are revealed in a σ_{A}-weighted mF_{obs}−DF_{calc} difference Fourier map (shown for metal B in green, contoured at 3.0σ) and in a Mn²⁺ anomalous difference Fourier map (shown for metal A in blue, contoured at 4.0σ). This figure was prepared with BOBSCRIPT and MOLSCRIPT.

FIG. 9. RNA exit and Rpb1 COOH-terminal repeat domain (CTD). (A) Previously proposed RNA exit grooves 1 and 2. The two grooves begin at the saddle between the clamp and wall and continue on either side of the Rpb1 dock region. The last ordered residue in Rpb1 (L1450) is indicated. The NH₂-terminal 25 residues of Rpb1 are highlighted in blue and correspond to an E. coli RNA polymerase fragment that was cross-linked to exiting RNA. The next 30 residues of Rpb1, which form the zipper, are highlighted in green and likely mark the location of E. coli residues that have been cross-linked to exiting RNA and to the upstream end of the transcription bubble. (B) Size and location of the CTD. The space available in the crystal lattice for the CTDs from four neighboring polymerases is indicated. The dashed line represents the length of a fully extended linker and CTD. The pink dashed circle indicates the size of a compacted random coil with the mass of the CTD.

FIG. 10. Proposed path for straight DNA in an initiation complex. (A) Top view. A B-DNA duplex was placed as indicated by the dashed cylinder. Rpb9 regions involved in start site selection are shown in orange. The location of mutations that affect initiation or start site selection are marked in yellow. The presumed location of general transcription factor TFIIB in a preinitiation complex is indicated by a dashed circle. (B) Back view. DNA may pass through the enzyme over the saddle between the wide open clamp (red) and the wall (blue). The circle corresponds in size to a B-DNA duplex viewed end-on.

FIG. 11. Sequence identity between RNA polymerases. (A) Residues identical in yeast and human Pol II sequences are highlighted in orange. (B) Residues identical in the corresponding yeast and E. coli sequences are highlighted in orange.

FIG. 12. A conserved RNA polymerase core structure. (A) Blocks of sequence homology between the two largest subunits of bacterial and eukaryotic RNA polymerases are in red. (B) Regions of structural homology between Pol II and bacterial RNA polymerase, as judged from a corresponding course of the polypeptide backbone, are in green.

FIG. 13. Nucleic acids in the transcribing complex and their interactions with pol II. (A) DNA (“tailed template”) and RNA sequences. DNA template and nontemplate strands are in blue and green, respectively, and RNA is in red. This color scheme is used throughout. (B) Ordering of nucleic acids in the transcribing complex structure. Nucleotides in the solid box are well ordered. Nucleotides in the dashed box are partially ordered, whereas those outside the boxes are disordered. Three protein regions that abut the downstream DNA are indicated. (C) Protein contacts to the ordered nucleotides boxed in (B). Amino acid residues within 4 Å of the DNA are indicated, colored according to the scheme for domain or domainlike regions of Rpb1 or Rpb2. Ribose sugars are shown as pentagons, phosphates as dots, and bases as single letters. Amino acid residues listed beside phosphates contact only this nucleotide. Amino acid residues listed beside riboses contact this nucleotide and its 3′-neighbor. Single-letter abbreviations for the amino acid residues are as follows: A, Ala; D, Asp; E, Glu; G, Gly; H, His; K, Lys; L, Leu; M, Met; N, Asn; Q, Gln; R, Arg; S, Ser; T, Thr; V, Val; and Y, Tyr. (D) Schematic representation of protein features participating in the detailed interactions shown in (C). Same notation as in (C), except that bases are shown as thick bars.

FIG. 14. Crystal structure of the pol II transcribing complex. (A) Electron density for the nucleic acids. On the left, the final sigma-weighted 2 mF_{obs}−DF_{calc} electron density for the downstream DNA duplex (dashed box in FIG. 13B) is contoured at 0.8σ (green). At this contour level, the surrounding solvent region shows only scattered noise peaks. A canonical 16-base pair B-DNA duplex was placed into the density. On the right, the final model of the DNA-RNA hybrid and flanking nucleotides (boxed in FIG. 1B) is superimposed on a simulated-annealing F_{obs}−F_{calc} omit map, calculated from the protein model alone with CNS (green, contoured at 2.6σ). The location of the active site metal A is indicated. (B) Comparison of structures of free pol II (top) and the pol II transcribing complex (bottom). The clamp (yellow) closes on DNA and RNA, which are bound in the cleft above the active center. The remainder of the protein is in gray. (C) Structure of the pol II transcribing complex. Portions of Rpb2 that form one side of the cleft are omitted to reveal the nucleic acids. Bases of ordered nucleotides (boxed in FIG. 1B) are depicted as cylinders protruding from the backbone ribbons. The Rpb1 bridge helix traversing the cleft is highlighted in green. The active site metal A is shown as a pink sphere.

FIG. 15. Switches, clamp loops, and the hybrid-binding site. (A) Stereoview of the clamp core (1, yellow) and the DNA and RNA backbones. The view is as in FIG. 14C. The five switches are shown in pink and are numbered. Three loops, which extend from the clamp and may be involved in transactions at the upstream end of the transcription bubble, are in violet. Major portions of the protein are omitted for clarity. (B) Stereoview of nucleic acids bound in the active center.

FIG. 16. Maintenance of the transcription bubble. (A) Schematic representation of nucleic acids in the transcribing complex. Solid ribbons represent nucleic acid backbones from the crystal structure. Dashed lines indicate possible paths of nucleic acids not present in the structure. (B) Protein elements proposed to be involved in maintaining the transcription bubble. Protein elements from Rpb1 and Rpb2 are shown in silver and gold, respectively.

FIG. 17. DNA-RNA hybrid conformation. The view is similar to that in FIG. 2C. The conformation of the DNA-RNA hybrid is intermediary between canonical A- and B-DNA. DNA, blue; RNA, red.

FIG. 18. Proposed transcription cycle and translocation mechanism. (A) Schematic representation of the nucleotide addition cycle. The nucleotide triphosphate (NTP) fills the open substrate site (top) and forms a phosphodiester bond at the active site (“Synthesis”). This results in the state of the transcribing complex seen in the crystal structure (middle). “Translocation” of the nucleic acids with respect to the active site (marked by a pink dot for metal A) may involve a change of the bridge helix from a straight (silver circle) to a bent conformation (violet circle, bottom). Relaxation of the bridge helix back to a straight conformation without movement of the nucleic acids would result in an open substrate site one nucleotide downstream and would complete the cycle. (B) Different conformations of the bridge helix in pol II and bacterial RNA polymerase structures. The view is the same as in FIG. 14C. The bacterial RNA polymerase structure was superimposed on the pol II transcribing complex by fitting residues around the active site. The resulting fit of the bridge helices of pol II (silver) and the bacterial polymerase (violet) is shown. The bend in the bridge helix in the bacterial polymerase structure causes a clash of amino acid side chains (extending from the backbone shown here) with the hybrid base pair at position +1.

FIG. 19. Stereo image of final α-amanitin structure. (A) σ_{A}-weighted F_{obs}−F_{calc} electron density at 2.8 Å resolution (red) contoured at 3 sigma calculated from the initial pol II placement before α-amanitin was included in the model. The final α-amanitin structure is shown (ball and stick model). (B) σ_{A}-weighted 2F_{obs}−F_{calc} electron density at 2.8 Å resolution (blue) contoured at 1.2 sigma, superimposed on the final α-amanitin structure (ball and stick model). Only the electron density around α-amanitin is shown. This figure was generated by using BOBSCRIPT and RASTER3D.

FIG. 20. Location of α-amanitin bound to pol II. (A) Cutaway view of a pol II-transcribing complex showing the location of α-amanitin binding (red dot) in relation to the nucleic acids and functional elements of the enzyme. (B) Ribbons representation of the pol II structure. Eight zinc atoms are shown in light blue, the active site magnesium is magenta, the region of Rpb1 around α-amanitin is light green (funnel) and dark green (bridge helix), the region of Rpb2 near α-amanitin is dark blue, and α-amanitin is red. This figure was prepared by using RIBBONS.

FIG. 21. Interaction of α-amanitin with pol II. (A) The chemical structure of π-amanitin, with residues of pol II that lie within 4 Å [determined by using CONTACT] placed near the closest contact. The Cαs of α-amanitin are labeled with blue numbers. Hydrogen bonds are shown as dashed lines with the distances indicated. (B) Stereoview of the α-amanitin binding pocket. Ball and stick models of α-amanitin (red bonds) and of pol II residues within 4 Å (gray bonds) are shown. Rpb1 from A700 to A809 (funnel region) is light green. Rpb1 from A810 to A825 (bridge helix) is dark green. Rpb2 from B760 to B769 is blue. This figure was generated by using BOBSCRIPT and RASTER3D.

FIG. 22. Complete, 12-subunit pol II electron density map. (A) Front view (as in ref. (10, 11)) of sigma-weighted FobS−Fcalc electron density at 4.1 Å resolution (green) contoured at 3 sigma, calculated from the initial placement of the pol II model (dark gray). The initial placement of archaeal RpoF (Rpb4 Homolog) is shown in red, and of archaeal RpoE (Rpb7 homolog) in blue. B) Electron density map at 4.1 Å resolution (yellow) contoured at 1.0 sigma, calculated using observed amplitudes (FobS) and phases after density modification. Superimposed is the final C-alpha Rpb4 (red) and Rpb7 (blue) model. This figure was generated using 0 and POV-ray (19).

FIG. 23A-B. Backbone model of complete, 12-subunit pol II. Ribbons representation of the complete pol II structure (“top” and “back” views). Rpb1 is gray, Rpb2 is bronze, Rpb4 is red, Rpb6 is green, the N-terminal half of Rpb7 which contains the RNP domain is dark blue, the C-terminal half of Rpb7 which contains the OB fold is light blue, and the remaining subunits are black. The locations of the clamp, the CTD, and the previously proposed RNA exit groove 1 (pink dashed line) are indicated. This figure was generated with Swiss-PDB viewer and POV-ray.

FIG. 24. Relationship of complete pol II X-ray structure to EM structures of (A) complete pol II (yellow map) and (B) Mediator-pol II complex (blue map). As this complex was prepared from exponentially growing yeast, it would have been largely deficient in Rpb4/Rpb7, accounting for the lack of density in this region of the EM map. The core pol II model is blue in A and yellow in B. Rpb4 is red and Rpb7 is dark blue. This figure was generated using O and POV-ray.

### DETAILED DESCRIPTION OF THE EMBODIMENTS

The present invention provides crystals and structures of an eukaryotic RNA polymerase, and an elongation complex containing a eukaryotic RNA polymerase. The structures and structural coordinates are useful in structural homology deduction, in developing and screening agents that affect the activity of eukaryotic RNA polymerase, and in designing modified forms of eukaryotic RNA polymerase. The structure information may be provided in a computer readable form, e.g. as a database of atomic coordinates, or as a three-dimensional model. The structures are useful, for example, in modeling interactions of the enzyme with DNA, RNA, transcription factors, nucleotides, etc. The structures are also used to identify molecules that bind to or otherwise interact with structural elements in the polymerase.

One aspect of the present invention provides crystals of the RNA polymerase II that can effectively diffract X-rays for the determination of the atomic coordinates of the RNA polymerase II to a resolution of better than 3.3 Angstroms, particularly where the polymerase includes nucleic acids involved in transcription. In another embodiment, the crystal effectively diffracts X-rays for the determination of the atomic coordinates of the RNA polymerase II to a resolution of 2.8 Angstroms or better. In a particular embodiment the RNA polymerase of the crystal is a yeast RNA polymerase II. Such a RNA polymerase comprises 10 subunits, and may further comprise nucleic acids involved in transcription, e.g. ribonucleotides, double stranded DNA, DNA-RNA hybrids, and mRNA. Also provided is a crystal of the complete 12-subunit enzyme, comprising the heterodimer of subunits Rpb4 and Rpb7, which associate reversibly with core. The RNA polymerase II may further comprise an inhibitor of transcription, e.g. α-amanitin. A crystal of the present invention may take a variety of forms all of which are included in the present invention.

The present invention further includes methods of using the structural information provided herein to derive a detailed structure of related polymerase enzymes, particularly other eukaryotic RNA polymerase II enzymes, which may be naturally occurring proteins, or variants thereof. Such structural homology determination may utilize modeling, alone or in combination with structure determination of the RNA polymerase.

The present invention provides three-dimensional coordinates for the RNA polymerase II structures, as deposited with the Protein Data Bank. Such a data set may be provided in computer readable form. Methods of using such coordinates (including in computer readable form) in drug assays and drug screens as exemplified herein, are also part of the present invention. In a particular embodiment of this type, the coordinates contained in the data set of can be used to identify potential modulators of the RNA polymerase II.

In one embodiment, a potential agent for modulation of RNA polymerase II is selected by performing rational drug design with the three-dimensional coordinates determined for the crystal. Preferably the selection is performed in conjunction with computer modeling. The potential agent is then contacted with the RNA polymerase II and the activity of the polymerase is determined. A potential agent is identified as an agent that affects the enzymatic activity or specificity of RNA polymerase II. Rational design may also be used in the genetic modification of RNA polymerase II, including any of its subunits, transcription factors, Mediator complex, etc., by modeling the potential effect of a change in the amino acid sequence of any of these polypeptides.

Computer analysis may be performed with one or more of the computer programs including: O (Jones et al. (1991) Acta Cryst. A47:110); QUANTA, CHARMM, INSIGHT, SYBYL, MACROMODEL; ICM, and CNS (Brunger et al. (1998) Acta Cryst. D54:905). In a further embodiment of this aspect of the invention, an initial drug screening assay, is performed using the three-dimensional structure so obtained, preferably along with a docking computer program. Such computer modeling can be performed with one or more Docking programs such as DOC, GRAM and AUTO DOCK. See, for example, Dunbrack et al. (1997) Folding & Design 2:2742.

It should be understood that in the drug screening and protein modification assays provided herein, a number of iterative cycles of any or all of the steps may be performed to optimize the selection. For example, assays and drug screens that monitor the activity of the RNA polymerase II in the presence and/or absence of a potential modulator (or potential drug) are also included in the present invention and can be employed as the sole assay or drug screen, or more preferably as a single step in a multi-step protocol.

### RNA Polymerase II Structure

The coordinates of the protein structures have been deposited at the Protein Data Bank (accession codes 1I3Q and 1I50 for the form 1 and form 2 structures, respectively). Elongation complex coordinates have been deposited at the Protein Data Bank (accession code 1I6H). See, Berman et al. (2000) Nucleic Acids Research 28:235-242 and Bernstein et al. (1977) J. Mol. Biol. 112:535-542. The coordinates of the 12 subunit complex have been deposited at PDB (accession code 1NIK). These coordinates can be used in the design of structural models and screening methods according to the methods of the invention.

Two crystal forms of the eukaryotic RNA polymerase II are provided. The crystal structures reveal the enzyme in two states: an open form and a partly closed form. These forms differ mainly in the position of a region of the enzyme called the clamp, which closes over the DNA as it enters the enzyme. A set of protein loops at the base of the clamp act as pivots for DNA movement. A structure is also provided for an actively transcribing complex of the enzyme with DNA. The electron density map shows the synthesized RNA, the DNA-RNA hybrid in the transcription bubble, and the three bases of the single-stranded DNA template that are unwound before it enters the hybrid duplex. The active site where the ester bond is broken in the substrate nucleoside triphosphates (NTPs) is marked by a metal ion at the base of the hybrid. The DNA double helix is situated in the cleft formed between the two largest enzyme subunits, Rpb1 and Rpb2. Structural elements described herein have been assigned names that explain their functions: wall, clamp, rudder, zipper. These structural elements do not directly correspond to protein domains because some of these elements may not fold independently.

As the DNA duplex enters the enzyme it is gripped by protein “jaws”. The 3′ (growing) end of the RNA is located adjacent to an active site Mg²⁺ ion. A “wall” of protein blocks the straight passage of nucleic acids through the enzyme, as a result of which the axis of the DNA-RNA makes almost a right angle with the axis of the entering DNA. The bend exposes the end of the DNA-RNA hybrid for addition of substrate nucleoside triphosphates (NTPs). The NTPs enter through a funnel-shaped opening on the underside of the enzyme and gain access to the active center through a pore. The 5′ end of the RNA abuts a loop of protein (the rudder), which prevents extension of the DNA-RNA hybrid beyond 9 base pairs, separating DNA from RNA. The exit path of the RNA passes beneath the rudder and beneath another loop of protein (the lid). The rudder and lid emanate from a massive clamp that swings over the active center region, restraining nucleic acids and contributing to the high processivity of transcription.

Translocation is accomplished with the help of a protein helix (the “bridge helix”) that spans the cleft between Rpb1 and Rpb2. Amino acid side chains from the bridge helix (threonine and alanine) make hydrophobic contacts with the base of the coding nucleotide in the template strand at the active site. This region is straight in the yeast polymerase II structure, but bent in the bacterial version by about 3 angstroms along the direction of the template strand. The bridge helix acts as a ratchet, allowing the release of the DNA and RNA strands for translocation but maintaining its grip on the growing end of the hybrid, thus enabling the next step in the elongation cycle to take place.

Also provided is the structure of the complete complex, which comprises the Rpb7 and Rpb4 heterodimer. Rpb7 interacts with both Rpb1 and Rpb6. A conserved region containing residues 15-20 makes a hydrophobic interaction with Ala 105 and Pro 106 of Rpb6. Residues corresponding to archaeal 55, 57, and 59 appear to be in a β-strand that adds to a β-sheet region of Rpb1 around Val 1443 to Ile 1445, beneath the previously described “RNA exit groove 1”. Residues 62 and 64 are in a loop penetrating the exit groove. Rpb7 contains an RNP fold and an OB fold. The OB fold is required for Rpb4/Rpb7 heterodimer binding to single stranded DNA and RNA. The heterodimer is placed near RNA exit groove 1, and interacts with RNA emanating from the groove. The surface of the triple-stranded β-sheet of the RNP fold, involved in RNA-binding in other examples of the fold, faces RNA exit groove 1. The RNP fold may serve to guide the transcript towards the OB fold, which lies about 50 Å from the exit of groove 1. A transcript length of 25-30 residues would be required to reach the OB-fold, and both capping of the 5′-end and a transition to a stable transcribing complex occur at about this length.

The N-terminal region of Rpb4 makes contact with the N-terminal region of Rpb1 around Ser 8 and Ala 9, located on the surface of the clamp above exit groove 1. Contacts of Rpb7 above the groove and Rpb4 below the groove bracket the clamp, constraining it in the closed state. The requirement for the heterodimer for the initiation of transcription and the effect of the heterodimer upon clamp closure suggest that promoter DNA binding and initiation occur in the clamp-closed state. Promoter DNA may bind to the enzyme in the clamp-open state, which affords a straight path through the active center cleft for unbent promoter DNA. In the clamp-closed state, promoter DNA may pass above the clamp and adjacent protein “wall”, descending into the active center region following melting and bending.

The location of the Rpb4/Rpb7 heterodimer in the complete enzyme suggests a role in the assembly of the transcription initiation complex. The heterodimer is adjacent to the site of TFIIB binding in a pol II-TFIIB cocrystal. Evidence for heterodimer-TFIIB interaction, stabilizing the transcription initiation complex, has come from surface plasmon resonance measurements. The location of the heterodimer in the complete enzyme in the vicinity of the C-terminal repeat domain (CTD) may be relevant to another interaction as well, that of Rpb4 with Fcp1, a phosphatase specific for the CTD.

The structure of complete pol II has implications for the mechanism of regulation by the multiprotein Mediator complex. Seven additional residues of Rpb1, which appear to interact with Rpb7, form part of the linker between the CTD and the body of pol II. The CTD is required for the binding of Mediator to pol II. The structure of a Mediator-pol II complex shows a crescent of Mediator density partly surrounding pol II. A gap between a “tail” region of the Mediator and the body of pol II, near the junction of the tail “middle” regions, corresponds to the location of the Rpb4/Rpb7 heterodimer in the X-ray structure, raising the possibility of direct Mediator-heterodimer interaction.

### Isolation and Crystallization of the RNA Polymerase

Crystals of the RNA polymerase of the present invention can be grown by a number of techniques including batch crystallization, vapor diffusion (either by sitting drop or hanging drop) and by microdialysis. Seeding of the crystals in some instances is required to obtain X-ray quality crystals. Standard micro and/or macro seeding of crystals may therefore be used. The crystals may be shrunk by transfer into solutions of different composition, e.g. by the addition of metal ions such as Mn²⁺, Pb²⁺, etc. Where the structure is to include nucleic acids, a DNA duplex bearing a single-stranded “tail” at one 3′-end may be included in the protein in order to generate a transcribing complex, usually in the absence of one of the four nucleoside triphosphates. Such a complex may be purified by passage through a column that binds the positively charged cleft of the enzyme, e.g. heparin columns. Crystals may also be generated that include inhibitors and other agents that interact with the protein, e.g. by soaking protein crystals in a solution comprising an inhibitor or other agent.

Supplemental crystals containing RNA polymerase II formed in the presence of the potential agent, or comprising altered polypeptides, may be made. Preferably the supplemental crystal effectively diffracts X-rays for the determination of the atomic coordinates to a resolution of better than 3.3 Angstroms, more preferably to a resolution equal to or better than 2.8 Angstroms. The three-dimensional coordinates of the supplemental crystal are then determined with molecular replacement analysis, which information may be used in the further design of agents and genetic modifications.

Alternative methods may also be used. For example, crystals can be characterized by using X-rays produced in a conventional source (such as a sealed tube or a rotating anode) or using a synchrotron source. Methods of characterization include, but are not limited to, precision photography, oscillation photography and diffractometer data collection. Selenium-methionine may be used as described in the examples provided herein, or alternatively a mercury derivative data set (e.g., using PCMB) may be used in place of the selenium-methionine derivatization.

Electron density maps may be built from crystals using phase information from multiple isomorphous heavy-atom derivatives. Model building is facilitated by the use of sequence markers, especially selenomethionine residues. Anomalous difference Fourier maps may be calculated with data from partially selenomethionine-substituted Pol II and with experimental multiple isomorphous replacement with anomalous scattering (MIRAS) phases (Hemming and Edwards (2000) J. Biol. Chem. 275:2288). Maps are improved by phase combination, where MIRAS phases are combined by the program SIGMAA (Jones et al., supra.) Phase combination may be followed by solvent flattening with DM (Carson (1997) Methods Enzymol. 277:493). Improved maps may be obtained by combination of the MIRAS phases with improved phases from combined polyalanine and atomic models in an iterative process. The model can be refined by classical positional and B-factor minimization, and with manual rebuilding.

### Structural Models and Databases

RNA polymerase II structure models and databases of structure information are provided. Models include structural data for the open and closed forms of RNA polymerase II; for an elongation complex comprising mRNA and RNA polymerase II, for a complex of RNA polymerase II with a bound inhibitor, and for the complete 12 subunit RNA polymerase II complex. Each of these models can be used independently for the rational design of drugs that affect cell proliferation, gene expression, transcriptional fidelity, specificity of antibiotics, and the like. Each of the models is also used in conjunction with the other models, for purposes of comparison of structural features, determining the effect of inhibitors, activators, RNA, and the like on the structure; for determining the role of specific subunits in RNA polymerase II function; and the like. Structural models of subunits and structural features can also be used independently, or in conjunction with other models. The structural models find use in determining the structure of related and/or homologous polymerase complexes, e.g. mammalian polymerase II, including human, mouse, monkey, etc. complexes. In some cases, modeling will be based on the provided polymerase II structure. In other embodiments, modeling will utilize the provided structure in combination with features present in homologous and/or related structures, where relationship may be defined by protein sequence similarity, or structural similarity, e.g. in the presence of specific features as described above.

The structure model may be implemented in hardware or software, or a combination of both. For most purposes, in order to use the structure coordinates generated for the structure, it is necessary to convert them into a three-dimensional shape. This is achieved through the use of commercially available software that is capable of generating three-dimensional graphical representations of molecules or portions thereof from a set of structure coordinates.

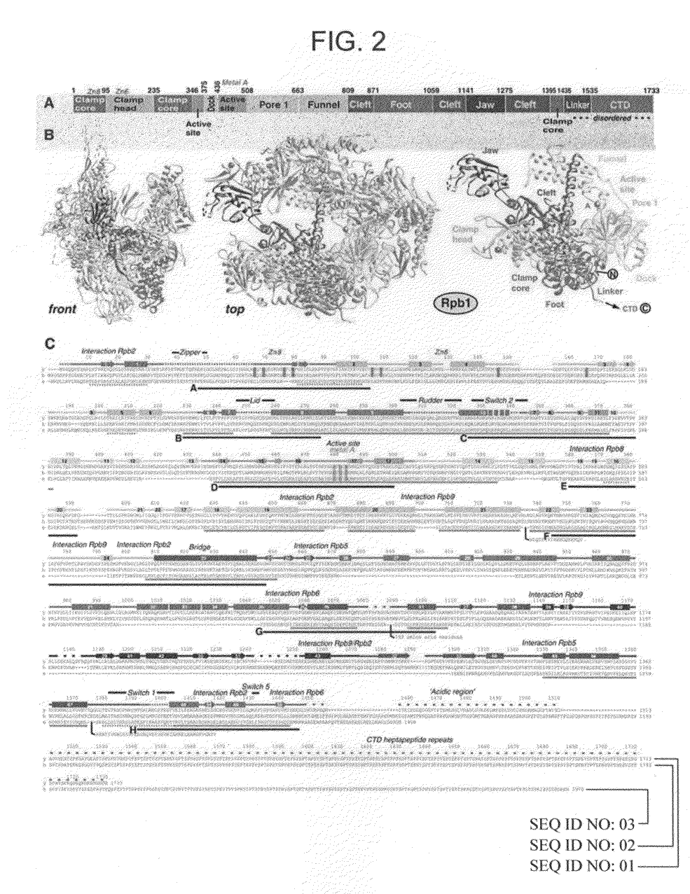

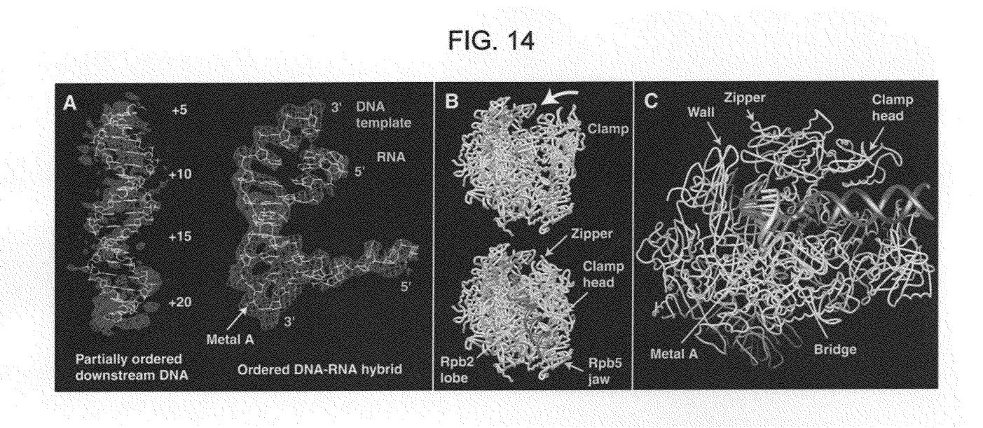

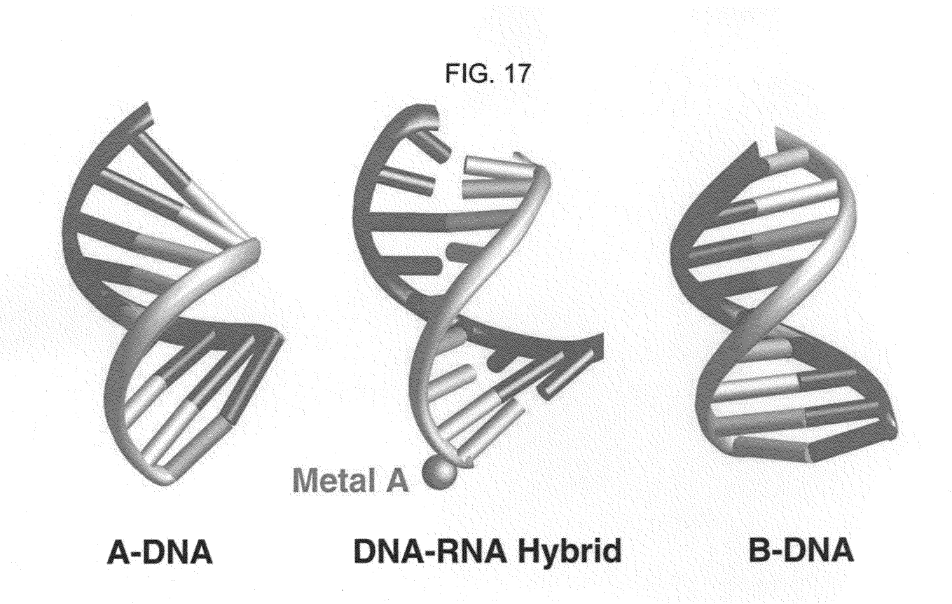

In one embodiment of the invention, a machine-readable storage medium is provided, the medium comprising a data storage material encoded with machine readable data which, when using a machine programmed with instructions for using said data, is capable of displaying a graphical three-dimensional representation of any of the structures of this invention that have been described above. Specifically, the computer-readable storage medium is capable of displaying a graphical three-dimensional representation of the RNA polymerase II protein, of an elongation complex comprising RNA polymerase II, of RNA polymerase II bound to an inhibitor, of the 12 subunit complete complex, or of specific structural elements in RNA polymerase II, which elements include the rudder, clamp core, clamp head, active site, pore 1, cleft, and funnel, as shown in FIG. 2D and the bridge, as shown in FIG. 14C and FIG. 17.

Thus, in accordance with the present invention, data providing structural coordinates, alone or in combination with software capable of displaying the resulting three dimensional structure of the enzyme, enzyme complex, and structural elements as described above, portions thereof, and their structurally similar homologues, is stored in a machine-readable storage medium. Such data may be used for a variety of purposes, such as drug discovery, analysis of interactions between cellular components during translation, modeling of vaccines, and the like.

Preferably, the invention is implemented in computer programs executing on programmable computers, comprising a processor, a data storage system (including volatile and non-volatile memory and/or storage elements), at least one input device, and at least one output device. Program code is applied to input data to perform the functions described above and generate output information. The output information is applied to one or more output devices, in known fashion. The computer may be, for example, a personal computer, microcomputer, or workstation of conventional design.

Each program is preferably implemented in a high level procedural or object oriented programming language to communicate with a computer system. However, the programs can be implemented in assembly or machine language, if desired. In any case, the language may be a compiled or interpreted language.

Each such computer program is preferably stored on a storage media or device (e.g., ROM or magnetic diskette) readable by a general or special purpose programmable computer, for configuring and operating the computer when the storage media or device is read by the computer to perform the procedures described herein. The system may also be considered to be implemented as a computer-readable storage medium, configured with a computer program, where the storage medium so configured causes a computer to operate in a specific and predefined manner to perform the functions described herein.

### Design of Binding Partners and Mimetics

The structure of the RNA polymerase II, complexes, and elements thereof, as described above, both independently and/or in combination are useful in the design of agents that modulate the activity and/or specificity of the enzyme, which agents may then alter patterns of transcription and gene expression. Agents of interest may comprise mimetics of the structural elements. Alternatively, the agents of interest may be binding agents, for example a structure that directly binds to a region of the polymerase II complex by having a physical shape that provides the appropriate contacts and space filling.

For example, the structure encoded by the data may be computationally evaluated for its ability to associate with chemical entities. This provides insight into an element's ability to associate with chemical entities. Chemical entities that are capable of associating with these domains may alter transcription. Such chemical entities are potential drug candidates. Alternatively, the structure encoded by the data may be displayed in a graphical format. This allows visual inspection of the structure, as well as visual inspection of the structure's association with chemical entities.

In one embodiment of the invention, a invention is provided for evaluating the ability of a chemical entity to associate with any of the molecules or molecular complexes set forth above. This method comprises the steps of employing computational means to perform a fitting operation between the chemical entity and the interacting surface of the polypeptide or nucleic acid; and analyzing the results of the fitting operation to quantify the association. The term “chemical entity”, as used herein, refers to chemical compounds, complexes of at least two chemical compounds, and fragments of such compounds or complexes.

Molecular design techniques are used to design and select chemical entities, including inhibitory compounds, capable of binding to an RNA polymerase II structural element. Such chemical entities may interact directly with certain key features of the structure, as described above. Such chemical entities and compounds may interact with one or more structural elements, in whole or in part.

It will be understood by those skilled in the art that not all of the atoms present in a significant contact residue need be present in a binding agent. In fact, it is only those few atoms which shape the loops and actually form important contacts that are likely to be important for activity. Those skilled in the art will be able to identify these important atoms based on the structure model of the invention, which can be constructed using the structural data herein.

The design of compounds that bind to or inhibit RNA polymerase II structural elements according to this invention generally involves consideration of two factors. First, the compound must be capable of either competing for bind with; or physically and structurally associating with the domains described above. Non-covalent molecular interactions important in this association include hydrogen bonding, van der Waals interactions, hydrophobic interactions and electrostatic interactions.

The compound must be able to assume a conformation that allows it to associate or compete with the RNA polymerase II structural element. Although certain portions of the compound will not directly participate in these associations, those portions of the may still influence the overall conformation of the molecule. This, in turn, may have a significant impact on potency. Such conformational requirements include the overall three-dimensional structure and orientation of the chemical entity in relation to all or a portion of the binding pocket, or the spacing between functional groups of an entity comprising several interacting chemical moieties.

Computer-based methods of analysis fall into two broad classes: database methods and de novo design methods. In database methods the compound of interest is compared to all compounds present in a database of chemical structures and compounds whose structure is in some way similar to the compound of interest are identified. The structures in the database are based on either experimental data, generated by NMR or x-ray crystallography, or modeled three-dimensional structures based on two-dimensional data. In de novo design methods, models of compounds whose structure is in some way similar to the compound of interest are generated by a computer program using information derived from known structures, e.g. data generated by x-ray crystallography and/or theoretical rules. Such design methods can build a compound having a desired structure in either an atom-by-atom manner or by assembling stored small molecular fragments. Selected fragments or chemical entities may then be positioned in a variety of orientations, or docked, within the interacting surface of the RNA. Docking may be accomplished using software such as Quanta (Molecular Simulations, San Diego, Calif.) and Sybyl, followed by energy minimization and molecular dynamics with standard molecular mechanics force fields, such as CHARMM and AMBER.

Specialized computer programs may also assist in the process of selecting fragments or chemical entities. These include: GRID (Goodford (1985) J. Med. Chem., 28, pp. 849-857; Oxford University, Oxford, UK; MCSS (Miranker et al. (1991) Proteins: Structure, Function and Genetics, 11, pp. 29-34; Molecular Simulations, San Diego, Calif.); AUTODOCK (Goodsell et al., (1990) Proteins: Structure, Function, and Genetics, 8, pp. 195-202; Scripps Research Institute, La Jolla, Calif.); and DOCK (Kuntz et al. (1982) J. Mol. Biol., 161:269-288; University of California, San Francisco, Calif.)

Once suitable chemical entities or fragments have been selected, they can be assembled into a single compound or complex. Assembly may be preceded by visual inspection of the relationship of the fragments to each other on the three-dimensional image displayed on a computer screen in relation to the structure coordinates. Useful programs to aid one of skill in the art in connecting the individual chemical entities or fragments include: CAVEAT (Bartlett et al. (1989) In Molecular Recognition in Chemical and Biological Problems”, Special Pub., Royal Chem. Soc., 78, pp. 182-196; University of California, Berkeley, Calif.); 3D Database systems such as MACCS-3D (MDL Information Systems, San Leandro, Calif.); and HOOK (available from Molecular Simulations, San Diego, Calif.).

Other molecular modeling techniques may also be employed in accordance with this invention. See, e.g., N. C. Cohen et al., “Molecular Modeling Software and Methods for Medicinal Chemistry, J. Med. Chem., 33, pp. 883-894 (1990). See also, M. A. Navia et al., “The Use of Structural Information in Drug Design”, Current Opinions in Structural Biology, 2, pp. 202-210 (1992).

Once the binding entity has been optimally selected or designed, as described above, substitutions may then be made in some of its atoms or side groups in order to improve or modify its binding properties. Generally, initial substitutions are conservative, i.e., the replacement group will have approximately the same size, shape, hydrophobicity and charge as the original group. It should, of course, be understood that components known in the art to alter conformation should be avoided. Such substituted chemical compounds may then be analyzed for efficiency of fit by the same computer methods described above.

Another approach made possible and enabled by this invention, is the computational screening of small molecule databases for chemical entities or compounds that can bind in whole, or in part, to the RNA polymerase II structural element. In this screening, the quality of fit of such entities to the binding site may be judged either by shape complementarity or by estimated interaction energy. Generally the tighter the fit, the lower the steric hindrances, and the greater the attractive forces, the more potent the potential modulator since these properties are consistent with a tighter binding constant. Furthermore, the more specificity in the design of a potential drug the more likely that the drug will not interact as well with other proteins. This will minimize potential side effects due to unwanted interactions with other proteins.

Compounds known to bind RNA polymerase II, for example alpha-amanitin, can be systematically modified by computer modeling programs until one or more promising potential analogs are identified. In addition systematic modification of selected analogs can then be systematically modified by computer modeling programs until one or more potential analogs are identified. Alternatively a potential modulator could be obtained by initially screening a random peptide library, for example one produced by recombinant bacteriophage. A peptide selected in this manner would then be systematically modified by computer modeling programs as described above, and then treated analogously to a structural analog.

Once a potential modulator/inhibitor is identified it can be either selected from a library of chemicals as are commercially available from most large chemical companies including Merck, GlaxoWelcome, Bristol Meyers Squib, Monsanto/Searle, Eli Lilly, Novartis and Pharmacia UpJohn, or alternatively the potential modulator may be synthesized de novo. The de novo synthesis of one or even a relatively small group of specific compounds is reasonable in the art of drug design.

### Biological Screening

The success of both database and de novo methods in identifying compounds with activities similar to the compound of interest depends on the identification of the functionally relevant portion of the compound of interest. For drugs, the functionally relevant portion may be referred to as a pharmacophore, i.e. an arrangement of structural features and functional groups important for biological activity. Not all identified compounds having the desired pharmacophore will act as a modulator of transcription. The actual activity can be finally determined only by measuring the activity of the compound in relevant biological assays. However, the methods of the invention are extremely valuable because they can be used to greatly reduce the number of compounds which must be tested to identify an actual inhibitor.

In order to determine the biological activity of a candidate pharmacophore it is preferable to measure biological activity at several concentrations of candidate compound. The activity at a given concentration of candidate compound can be tested in a number of ways. The physical interactions are tested by combining the RNA polymerase II, or a fragment thereof with the candidate compound.

For example, the RNA polymerase II can be attached to a solid support. Methods for placing proteins on a solid support are well known in the art and include such steps as linking biotin to the protein, and linking avidin to the solid support. The solid support can be washed to remove unreacted species. A solution of a labeled potential modulator (e.g., an inhibitor) can be contacted with the solid support. The solid support is washed again to remove the potential modulator not bound to the support. The amount of labeled potential modulator remaining with the solid support and thereby bound to the enzyme can be determined Alternatively, or in addition, the dissociation constant between the labeled potential modulator and the enzyme, for example can be determined.

In another embodiment, a Biacore machine can be used to determine the binding constant of the RNA polymerase II to a DNA template in the presence and absence of the potential modulator. Alternatively, one or more of the RNA polymerase subunits can be immobilized on a sensor chip. The remaining subunits can then be contacted with (e.g. flowed over) the sensor chip to form the RNA polymerase. The dissociation constant for the RNA polymerase can be determined by monitoring changes in the refractive index with respect to time as buffer is passed over the chip. Scatchard Plots, for example, can be used in the analysis of the response functions using different concentrations of a particular subunit. Flowing a potential modulator at various concentrations over the RNA polymerase II and monitoring the response function (e.g., the change in the refractive index with respect to time) allows the dissociation constant to be determined in the presence of the potential modulator and thereby indicates whether the potential modulator is either an inhibitor, or an agonist of the enzyme complex.

In another aspect of the present invention a potential modulator is assayed for its ability to inhibit the RNA polymerase II. A modulator that inhibits the RNA polymerase can then be selected. In a particular embodiment, the effect of a potential modulator on the catalytic activity of RNA polymerase II is determined. The potential modulator is then added to a cell sample to determine its effect on proliferation. A potential modulator that inhibits proliferation can then be selected.

The effect of the potential modulator on the catalytic activity of the RNA polymerase II may be determined (either independently, or subsequent to a binding assay as exemplified above). In one such embodiment, the rate and/or specificity of the DNA-dependent RNA transcription is determined. For such assays a labeled nucleotide could be used. This assay can be performed using a real-time assay, e.g. with a fluorescent analog of a nucleotide. Alternatively, the determination can include the withdrawal of aliquots from the incubation mixture at defined intervals and subsequent placing of the aliquots on nitrocellulose paper or on gels.

It is to be understood that this invention is not limited to the particular methodology, protocols, animal species or genera, constructs, and reagents described, as such may vary. It is also to be understood that the terminology used herein is for the purpose of describing particular embodiments only, and is not intended to limit the scope of the present invention, which will be limited only by the appended claims.

As used herein the singular forms “a”, “and”, and “the” include plural referents unless the context clearly dictates otherwise. Thus, for example, reference to “an immunization” includes a plurality of such immunizations and reference to “the cell” includes reference to one or more cells and equivalents thereof known to those skilled in the art, and so forth. All technical and scientific terms used herein have the same meaning as commonly understood to one of ordinary skill in the art to which this invention belongs unless clearly indicated otherwise.

### EXPERIMENTAL

### Example 1

### RNA Polymerase at 2.8 Å Resolution

Structures of a 10-subunit yeast RNA polymerase II have been derived from two crystal forms at 2.8 and 3.1 angstrom resolution. Comparison of the structures reveals a division of the polymerase into four mobile modules, including a clamp, shown previously to swing over the active center. In the 2.8 angstrom structure, the clamp is in an open state, allowing entry of straight promoter DNA for the initiation of transcription. Three loops extending from the clamp may play roles in RNA unwinding and DNA rewinding during transcription. A 2.8 angstrom difference Fourier map reveals two metal ions at the active site, one persistently bound and the other possibly exchangeable during RNA synthesis. The results also provide evidence for RNA exit in the vicinity of the carboxyl-terminal repeat domain, coupling synthesis to RNA processing by enzymes bound to this domain.

Presented here are atomic structures determined from the previous crystal form at 3.1 Å resolution and from a new crystal form, containing the enzyme in a different conformation, at 2.8 Å resolution. The structures illuminate the transcription mechanism. They provide a basis for understanding both transcription initiation and RNA chain elongation. They permit the identification of protein features and amino acid residues crucial in the structure of an actively transcribing complex.

Atomic structures of Pol II. The Pol II crystals from which the previous backbone model was derived were grown and then shrunk by transfer to a solution of different composition (Cramer et al. (2000) Science 288, 640). Shrinkage reduced the a axis of the unit cell by 11 Å and improved the diffraction from about 6.0 to 3.0 Å resolution (crystal form 1). It was subsequently found that addition of Mn²⁺, Pb²⁺, or other metal ions induced a further shrinkage by 8 Å along the same unit cell direction and improved diffraction to 2.6 Å resolution in favorable cases (crystal form 2, Table 1). Addition of 1 to 10 mM Mg2+, Mn2+, Pb2+, or lanthanide ions led to further shrinkage. The resulting form 2 crystals had a slightly lower solvent content and lower mosaicity. Shrinkage of form 1 to form 2 results in additional crystal contacts of the mobile clamp and jaw-lobe module (see below), which may account for the improvement in diffraction. Differences in Pol II conformation between form 1 and form 2, as well as atomic details most visible in form 2, led to the conclusions reported here.

TABLE 1
Crystallographic data and structure statistics.

| | Crystal form | |
|---|---|---|
| | 1 | 2 |
| Data collection-* | | |
| Space group | I222 | I222 |
| Unit cell dimensions (Å) | 130.7 by 224.8 by 369.4 | 122.7 by 223.0 by 376.1 |
| Wavelength (Å) | 1.283^{†} | 1.291^{†} |
| Resolution (Å) | 40-3.1 (3.2-3.1)^{‡} | 40-2.8 (2.9-2.8)^{‡} |
| Unique reflections | 98,315 (9,073)^{‡} | 125,251 (12,023)^{‡} |
| Completeness (%) | 99.2 (92.7)^{‡} | 99.0 (96.2)^{‡} |
| Redundancy | 4.7 | 3.6 |
| Mosaicity (°) | 0.44 | 0.36 |
| R_{sym} (%)^{§} | 8.4 (29.8)^{‡} | 5.8 (34.4)^{‡} |
| Refinement | | |
| Nonhydrogen atoms | 28,173 | 28,379 |
| Protein residues | 3543 | 3559 |
| Water molecules | 0 | 78 |
| Metal ions | 8 Zn²⁺, 1 Mg²⁺ | 8 Zn²⁺, 1 Mn²⁺ |
| Anisotropic scaling (B₁₁, B₂₂, B₃₃) | _7.9, 11.3, 6.7 | _14.2, 4.3, 9.9 |
| rmsd bonds (Å) | 0.008 | 0.007 |
| rmsd angles (°) | 1.50 | 1.43 |
| Reflections in test set (%) | 4,778 (4.8) | 3,800 (3.0) |
| R_{cryst}/R_{free}^{\|\|} | 22.9/28.3 | 22.9/28.2 |

*Data for form 1 are from Cramer et al. (2000), supra. Data collection for form 2 was carried out at 100 K as described in Cramer et al. with an ADSC Quantum 4 charge-coupled device detector at beamline 9-2 of SSRL. Diffraction data were processed with DENZO and SCALEPACK (79).
^{†}Data for form 1 were collected at the Zn²⁺ anomalous peak to reveal native Zn²⁺ sites. Data for form 2 were collected below the Zn²⁺ anomalous peak energy to localize the Mn²⁺ ion at the active center.
^{‡}Values in parentheses correspond to the highest resolution shells.
^{§}R_{sym} = Σ_{i,h}|I(i, h) ₋ [symbol] (h) [symbol] /Σ_{i,h}|I(i, h)|, where [symbol] (h) [symbol] is the mean of the I observations of reflection h. R_{sym} was calculated with anomalous pairs merged; no σ cut-off was applied.
^{||}R_{cryst/free} = Σₕ||F_{obs}(h)| ₋ |F_{calc}(h)||/Σₕ|F_{obs}(h)|. R_{cryst} and R_{free} were calculated from the working and test reflection set, respectively.

An atomic model was initially built in electron density maps from crystal form 1, for which phase information from multiple isomorphous heavy-atom derivatives was available. Model building was facilitated by the use of sequence markers, especially 94 selenomethionine residues, and maps were gradually improved by phase combination. A total of 141 amino acid residues were located by sequence markers. Out of 103 methionine residues in the final structure, 94 were revealed as peaks of greater than 3.3 in a 4 Å anomalous difference Fourier map calculated with data from partially selenomethionine-substituted Pol II and with experimental multiple isomorphous replacement with anomalous scattering (MIRAS) phases. The few remaining methionines are located in poorly ordered regions. In the selenomethionine-substituted Pol II map, three cysteine residues, C520 and C1400 in Rpb1 and C207 in Rpb3, also showed peaks. Eight Zn2+ ions confirmed the location of 31 cysteine residues and one histidine residue (FIGS. 2 to 5). The active-site metal A is coordinated by three invariant aspartate residues in Rpb1 (FIG. 2). Two different Hg derivatives revealed the location of 10 surface cysteine residues (Rpb1, C1400, C1421; Rpb2, C64, C302, C388, C533; Rpb3, C207; Rpb5, C83; Rpb8, C24, C36). MIRAS phases were combined by the program SIGMAA with phases from the initial polyalanine model. Phase combination was followed by solvent flattening with DM. This led to an electron density map at 3.1 Å resolution in which many side chains were visible. Improved maps were obtained by combination of the MIRAS phases with improved phases from combined polyalanine and atomic models in an iterative process.

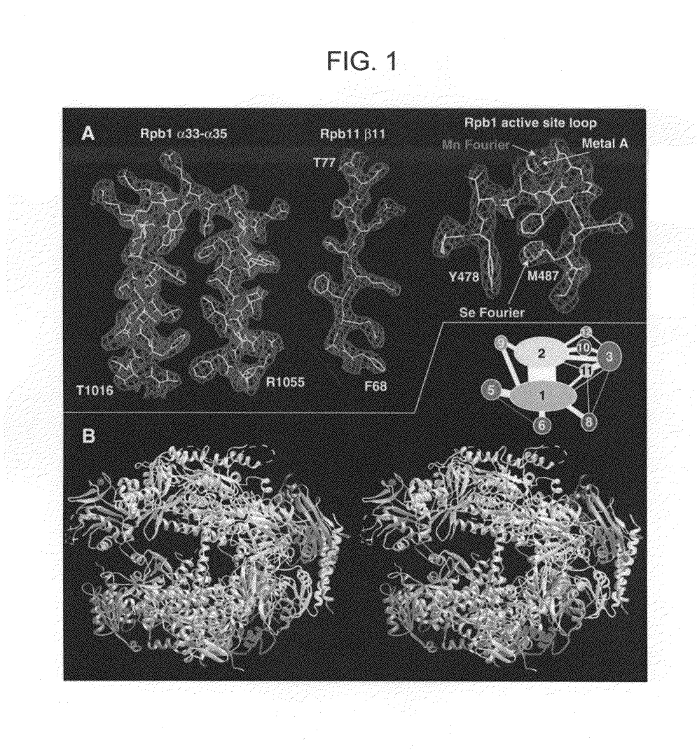

The model was refined at 3.1 Å resolution by classical positional and B-factor minimization, alternating with manual rebuilding. Model building was carried out with the program O, and refinement, with the program CNS. After bulk solvent correction and anisotropic scaling, the model was subjected to positional minimization in CNS with experimental phase restraints (MLHL target). After several rounds of model building into the resulting A-weighted electron density maps and subsequent further refinement, the maximum likelihood target function (MLF) was used and restrained atomic B-factor refinement was carried out. With the resulting phase-combined maps, poorly ordered regions such as parts of the clamp and the Rpb2 lobe region could be built. Extensive rebuilding and refinement of atomic positions and B factors lowered the free R factor to 29.8%. Inclusion in the form 1 structure of fine stereochemical adjustments that were achieved in refinement of the form 2 structure lowered the free R factor to 28.3%. The resulting structure was placed in crystal form 2 and further refined at 2.8 Å resolution to a free R factor of 28.2% (Table 1). The form 1 structure was manually placed with experimental Zn²⁺-ion positions and the position of the active-site metal in form 2. The clamp was adjusted to its new position relative to the rest of Pol II. After initial rigid body refinement of the entire polymerase in CNS, A-weighted difference electron density maps revealed regions that had moved. Manual adjustment of these regions was followed by rigid body refinement in groups and positional and atomic B-factor refinement. The structure in form 2 was further confirmed with the use of sequence markers, including selenomethionine. After several rounds of fine adjustment of the model stereochemistry and further refinement, 78 water molecules could be included. Electron density maps at that resolution revealed side-chain conformations and the orientations of backbone carbonyl groups (FIG. 1A).

Both form 1 and form 2 structures contain over 3500 amino acid residues, with more than 28,000 nonhydrogen atoms and 8 Zn²⁺ ions (Table 1). The Mg²⁺ ion in form 1 is replaced by a Mn²⁺ ion in form 2, and several additional loops, as well as 78 structural water molecules, are also seen in form 2. The stereochemical quality of the structures is high, with 98.0% of the residues in form 2 in allowed regions of the Ramachandran plot, and all residues in disallowed regions located in mobile loops for which only main-chain density was observed. Disordered regions in the structures are limited to the COOH-terminal repeat domain (CTD) of the largest subunit, Rpb1, to the nonconserved NH₂-terminal tails of Rpb6 and Rpb12, and to several short exposed loops in Rpb1, Rpb2, and Rpb8.

Regions showing only main-chain electron density: Rpb1, amino acids 1 to 4, 36 to 66, 154 to 157, 186 to 197, 248 to 266, 307 to 323, 330 to 338, 1388 to 1403; Rpb2, 69 to 70, 133 to 138, 241 to 251, 434 to 437, 643 to 649, 864 to 872, 915 to 919, 933 to 935, 1104 to 1110; Rpb5, 1 to 5; Rpb8, 29 to 35, 82 to 91, 107 to 113, 127 to 139; Rpb9, 1 to 4, 116 to 122; Rpb12, 24 to 53.

Disordered regions: Rpb1, amino acids 1082 to 1091, 1177 to 1186, 1244 to 1253, 1451 to 1733; Rpb2, 1 to 17, 71 to 88, 139 to 163, 438 to 445, 468 to 476, 503 to 508, 669 to 677, 713 to 721, 920 to 932, 1111 to 1126; Rpb3, 1 to 2, 269 to 318; Rpb6, 1 to 71; Rpb8, 1, 64 to 75; Rpb10, 66 to 70, Rpb11, 115 to 120; Rpb12, 1 to 23.

Over 53,000 Å² of surface area is buried in subunit interfaces (FIG. 1B and Table 2), about a third of it between Rpb1 and Rpb2, accounting for the high stability of Pol II. Many salt bridges and hydrogen bonds, and some structural water molecules, five at 2.8 Å resolution, are observed in the interfaces. There are seven instances of a “β-addition motif,” in which a strand from one subunit is added to a β sheet of another. The COOH-terminal region of Rpb12, which bridges between Rpb2 and Rpb3, participates in two such β-addition motifs (Table 2). The importance of one of these motifs is shown by deletion of two residues from the COOH-terminus of Rpb12, which confers a lethal phenotype. Termini of Rpb10 and Rpb11 also play structural roles, whereas the remaining 17 subunit termini extend outwards into solvent.

The NH2-terminal methionine of Rpb10 is inserted in a hydrophobic pocket lined by Rpb2, Rpb3, and Rpb11. The NH2-terminus of Rpb11 binds in the previously proposed RNA exit groove 2. The charge of its terminal amino group is neutralized by the conserved residue D100 of Rpb2. The COOH-terminal residue R70 of Rpb12 is linked by a salt-bridge to the conserved residue E166 of Rpb3, whereas the charge of its carboxylate is neutralized by the conserved residue R852 of Rpb2.

TABLE 2
Subunit interactions.

| Subunit | Buried surface | | Hydrogen | |
|---|---|---|---|---|
| interface | area (Å²)-* | Salt bridges^{†} | bonds^{‡} | β-addition motifs^{§} |
| Rpb1-Rpb2 | 17,178 | 6 | 58 | Rpb2-β41-Rpb1-β7; Rpb2- |
| | | | | β45-Rpb1-β1 |
| Rpb1-Rpb3 | 608 | 1 | 3 | — |
| Rpb1-Rpb5 | 4,768 | 5 | 19 | — |
| Rpb1-Rpb6 | 3,797 | 3 | 12 | Rpb1-β35-Rpb6-β3 |
| Rpb1-Rpb8 | 3,056 | 3 | 6 | Rpb8-β6-Rpb1-β18 |
| Rpb1-Rpb9 | 3,011 | 2 | 21 | Rpb9-β4-Rpb1-β28 |
| Rpb1-Rpb11 | 1,913 | — | 8 | — |
| Rpb2-Rpb3 | 3,070 | 5 | 26 | — |
| Rpb2-Rpb9 | 2,705 | 1 | 5 | — |
| Rpb2-Rpb10 | 2,941 | 1 | 11 | — |
| Rpb2-Rpb11 | 608 | 1 | 2 | — |
| Rpb2-Rpb12 | 1,923 | 4 | 14 | Rpb12-β3-Rpb2-β32 |
| Rpb3-Rpb8 | 333 | 1 | 1 | — |
| Rpb3-Rpb10 | 2,175 | 4 | 15 | — |
| Rpb3-Rpb11 | 3,899 | 4 | 6 | — |
| Rpb3-Rpb12 | 993 | 3 | 7 | Rpb12-β4-Rpb3-β3 |
| Rpb5-Rpb6 | 204 | 1 | 3 | — |
| Rpb8-Rpb11 | 396 | — | — | — |
| Total | 53,578 | 45 | 217 | 7 instances |

*Calculated with programs AREAIMOL and RESAREA with a standard probe radius of 1.4 Å.
^{†}A conservative distance cut-off of 3.6 Å was used [program CONTACT].
^{‡}Potential hydrogen bonds with a donor-acceptor distance below 3.3 Å were included.
^{§}The order of strands in a β-addition motif is added β strand-accepting strand of a β sheet. Biochemical mapping suggests that the β-addition motif formed by Rpb1 and Rpb9 may be largely responsible for the interaction of these subunits. The β-addition motif formed between Rpb1 and Rpb6 restrains clamp mobility.

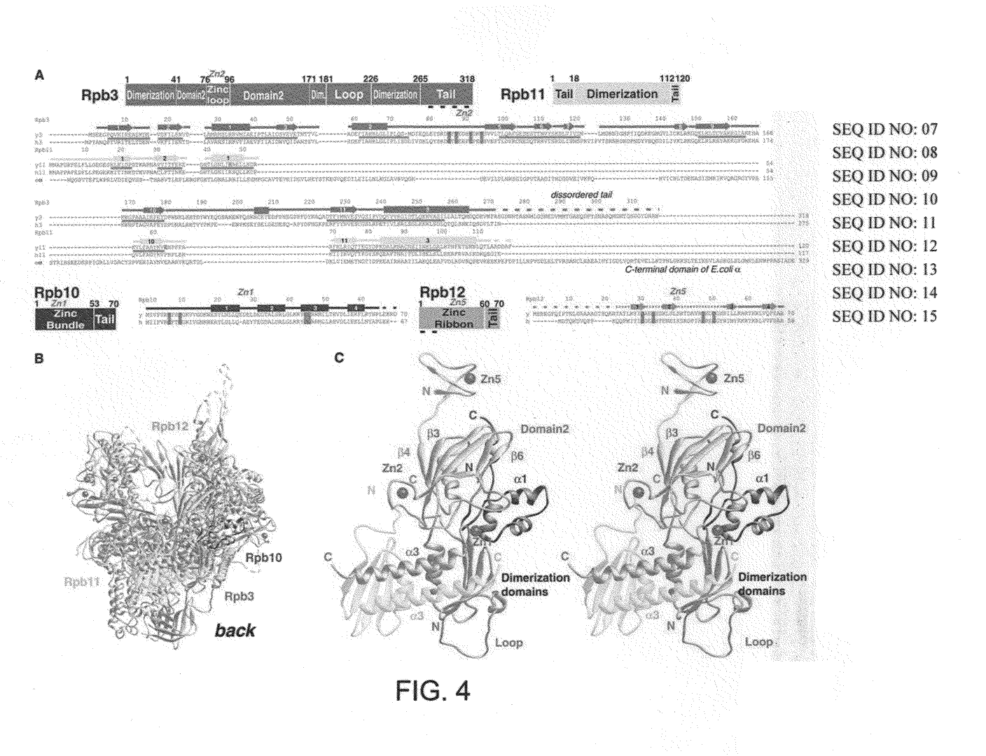

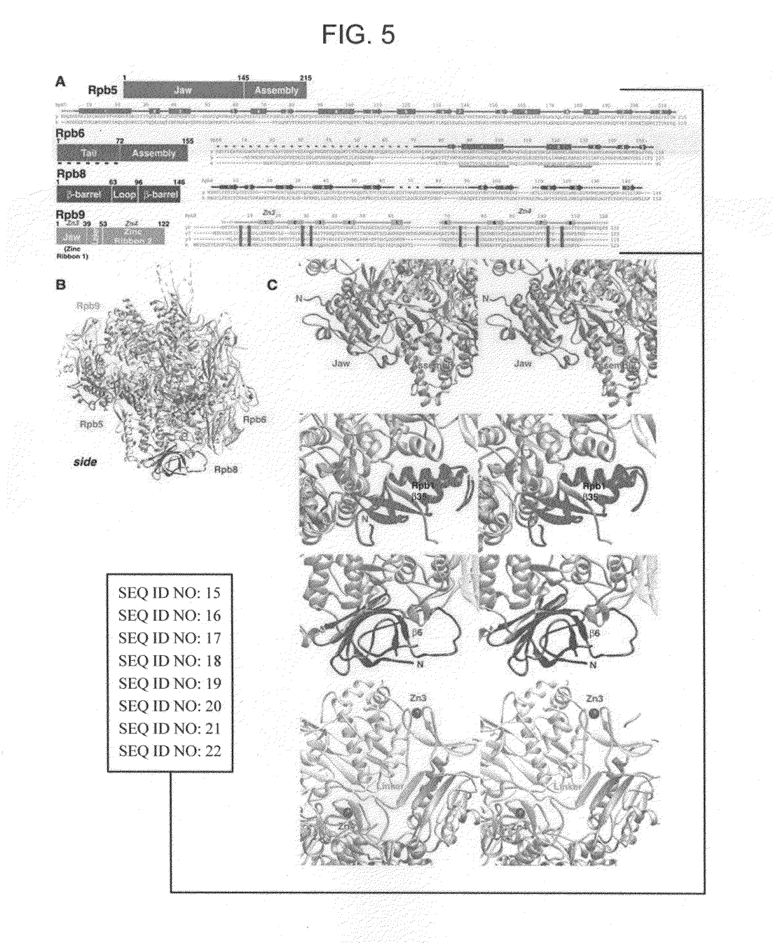

For ease of display and discussion, all Pol II subunits are represented as arrays of domains or domainlike regions, named according to their locations or presumed functional roles (FIGS. 2 to 5). In many cases, however, these domains and regions do not appear to be independently folded. For example, the “active site” region of Rpb1 and the “hybrid-binding” region of Rpb2 combine in a single fold that forms the active center of the enzyme (FIGS. 1B, 2, and 3). None of the folds in Rpb1 and Rpb2 could be found in the protein structure database and so all are evidently unique. Domains and domainlike regions of Rpb1 and Rpb2 did not produce any significant matches when submitted to the DALI server. The unique folds of the large subunits appear to depend on extensive contacts with small subunits on the periphery (Table 2). Rpb3, Rpb5, and Rpb9 each consist of two independent domains, whereas the remaining small subunits form single domains (FIGS. 4 and 5).

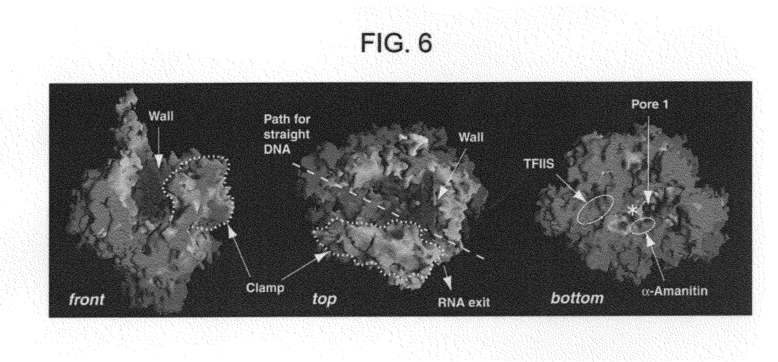

The surface charge of Pol II is almost entirely negative, except for a uniformly positively charged lining of the cleft, the active center, the wall, and a “saddle” between the clamp and the wall (FIG. 6). This strongly asymmetric charge distribution accords with previous proposals for the paths of DNA and RNA in a transcribing complex. It is also consistent with previous evidence for an electrostatic component of the polymerase-DNA interaction. The positively charged environment of the cleft may help to localize DNA without restraining movement toward the active site for transcription. The positive charge on the saddle supports the proposal that it serves as an exit path for RNA. Homology modeling of human Pol II reveals that the overall surface charge distribution is well conserved.

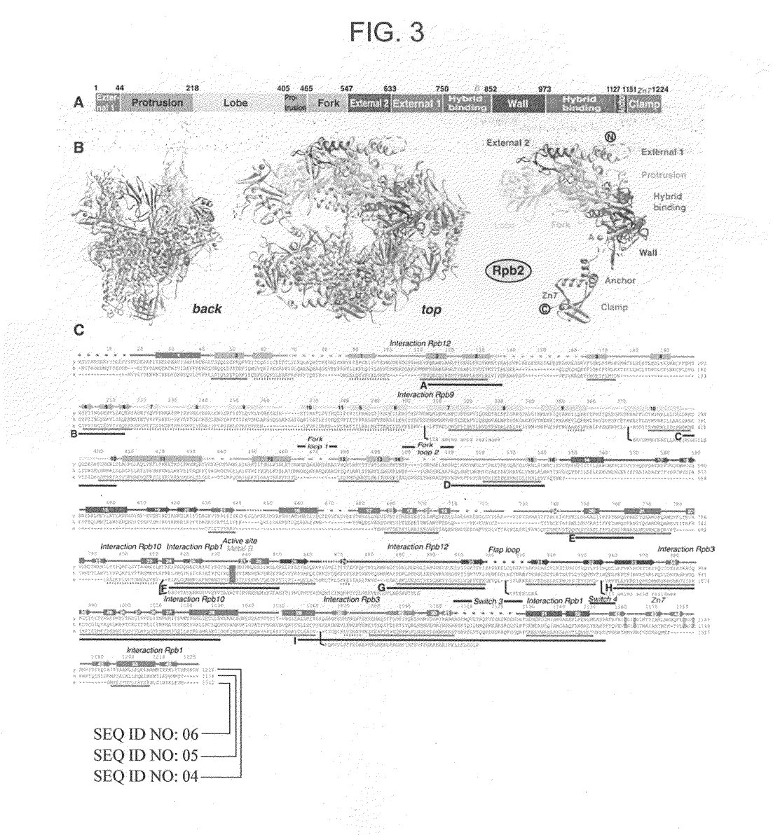

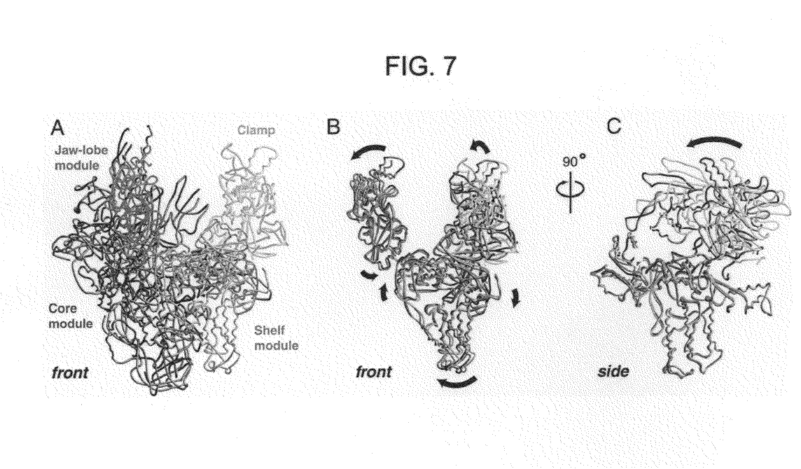

Four mobile modules. Comparison of the form 1 and form 2 structures reveals a division of the polymerase into four mobile modules (FIG. 7 and Table 3). Half the mass of the enzyme lies in a “core” module, containing the regions of Rpb1 and Rpb2 that form the active center and subunits Rpb3, Rpb10, Rpb11, and Rpb12, which have been implicated in Pol II assembly. Three additional modules, whose positions relative to the core module change between form 1 and form 2, lie along the sides of the DNA-binding cleft, before the active center. The “jaw-lobe” module contains the “upper jaw”, made up of regions of Rpb1 and Rpb9, and the “lobe” of Rpb2 (FIGS. 3 and 4). The “shelf” module contains the “lower jaw” (a domain of Rpb5), the “assembly” domain of Rpb5, Rpb6, and the “foot” and “cleft” regions of Rpb1 (FIG. 3 and FIG. 4). The remaining module, the “clamp,” was originally identified as a mobile element in a Pol II map at 6 Å resolution.

TABLE 3
Mobile modules.

| | | Percentage of | Maximum Cα atom displacement |
|---|---|---|---|
| Module | Subunits and regions | total mass | (Å) (residue number) |
| Core | All except other three | 57 | — |
| | modules | | |
| Shelf | Rpb1 cleft, Rpb1 foot, Rpb5, | 21 | 3.3 (N903 of Rpb1) |
| | Rpb6 | | |
| Clamp | Rpb1 clamp core and clamp | 12 | 14.2 (D193 of Rpb1); 14.4 (G283 |
| | head, Rpb2 clamp | | of Rpb1) |
| Jaw- | Rpb1 jaw, Rpb9 jaw, Rpb2 | 10 | 4.3 (K347 of Rpb2) |
| lobe | lobe | | |

The changes observed between form 1 and form 2 structures are small rotations of the jaw-lobe and shelf modules about axes roughly parallel to the cleft (perpendicular to the plane of the page in FIG. 7B), producing movements of individual amino acid residues of up to 4 Å, and a larger swinging motion of the clamp, resulting in movements of as much as 14 Å (Table 3). The mobility of the clamp is also evidenced by its high overall temperature factor (Table 4). Rotations of the jaw-lobe and shelf modules may contribute to a helical screw rotation of the DNA as it advances toward the active center.

TABLE 4
Crystallographic temperature factors.

Average atomic B factor (Å²)

| Selection of model atoms | Crystal form 1 | Crystal form 2 |
|---|---|---|
| Rpb1 | 71.8 | 64.0 |
| Rpb2 | 70.4 | 61.5 |
| Rpb3 | 59.1 | 59.5 |
| Rpb5 | 78.6 | 69.1 |
| Rpb6 | 59.5 | 51.8 |
| Rpb8 | 101.7 | 100.0 |
| Rpb9 | 75.1 | 67.6 |
| Rpb10 | 57.6 | 51.2 |
| Rpb11 | 56.2 | 62.0 |
| Rpb12 | 108.0 | 97.7 |
| Clamp | 113.3 | 81.6 |
| Water | — | 39.4 |
| Molecules | | |
| Active-site metal A | 58.4 (Mg²⁺) | 40.7 (Mn²⁺) |
| Zn²⁺ ions | 119.1 | 84.9 |
| Overall | 71.5 | 64.5 |

The swinging motion of the clamp produces a greater opening of the cleft in form 2 than form 1, which may permit the entry of promoter DNA for the initiation of transcription (see below). Features seen in the form 2 structure suggest that, upon closure in a transcribing complex, the clamp serves as a multifunctional element, sensing the DNA-RNA hybrid conformation and separating DNA and RNA strands at the upstream end of the transcription bubble. The unique clamp fold is formed by NH₂— and COOH-terminal regions of Rpb1 and the COOH-terminal region of Rpb2. At the base of the clamp, these regions are held together in a β sheet made up of one strand from each region (Rpb1 β1, Rpb1 β34, and Rpb2 β46). Not included at the base of the clamp is the NH₂-terminal tail of Rpb6, the only change in subunit assignment of a density feature between the atomic structures and the previous backbone model. Incorporation of the Rpb6 tail in the backbone model was based on early electron density maps and the NMR structure of free Rpb6. Several residues in the NH₂-terminal tail form an outer strand of a β sheet in the NMR structure. In the course of building the previous Pol II backbone model, the NMR structure was placed in the available electron density and the outer strand of the Rpb6, sheet was extended toward the NH₂-terminus, following continuous density into the base of the clamp. The current, improved maps and sequence markers show that the continuous density near the base of the clamp instead corresponds to part of conserved region H of Rpb1, and that the NH₂-terminal tail of Rpb6 is disordered. It is stabilized by three Zn²⁺ ions, two within the “clamp core” and one underlying a distinct region at the upper end, termed the “clamp head”. Zinc ions Zn7 and Zn8 in the clamp core are bound by residues in the common motif CX₂CXₙCX₂C/H (where X is any amino acid). Zinc ion Zn6 shows an unusual coordination that underlies the clamp head fold (FIG. 2).

Mutations of the Zn²⁺-coordinating cysteine residues in the clamp confer a lethal phenotype. At its base, the clamp is connected to the “cleft” region of Rpb1, to the “anchor” region of Rpb2, and to Rpb6 through a set of “switch” regions that are flexible and enable clamp movement (FIGS. 2 and 3). Whereas the shorter switches (4 and 5) are well ordered, the longer switches are poorly ordered (switches 1 and 2) or disordered (switch 3). All five switches undergo conformational changes in the transition to a transcribing complex, and switches 1, 2, and 3 contact the DNA-RNA hybrid in the active center. The switches therefore couple closure of the clamp to the presence of the DNA-RNA hybrid, which is key to the processivity of transcription. Interaction with the DNA-RNA hybrid may also be instrumental in the readout of the template DNA sequence in the active center.

Weak electron density is seen for three loops extending from the clamp that may interact with DNA and RNA upstream of the active-center region. The loop nearest the active center corresponds to a “rudder” previously noted in the structure of bacterial RNA polymerase and suggested to participate in the separation of RNA from DNA and maintenance of the upstream end of the RNA-DNA hybrid. The rudder, corresponding to Rpb1 residues 304 to 324, was not detected in early electron density maps of Pol II and so is absent from the previous backbone model of Pol II. Main-chain density for the rudder is clearly revealed in the improved, phase-combined electron density maps reported here. The second and third loops, here termed “lid” and “zipper” (FIG. 2D, “Clamp core, Linker,” viewed in stereo), may be involved in these processes as well. Although disordered in the bacterial polymerase structure, both lid and zipper are apparently conserved. The lid and zipper are located in sequence homology blocks B and A, respectively. The lid is also flanked by regions of conserved structure. They lie 10 to 20 Å, corresponding to roughly three to six nucleotides, beyond the rudder. The rudder and lid may be involved in the separation of RNA from DNA, whereas the lid and zipper maintain the upstream end of the transcription bubble. In keeping with this idea, a region in the largest subunit of the Escherichia coli enzyme containing residues corresponding to the zipper has been cross-linked to the upstream end of the bubble. A disordered loop on top of the wall, termed the “flap loop” (FIG. 3), may cooperate with the lid and zipper in the maintenance of the bubble. The region termed the “wall” in Pol II corresponds to a feature referred to as the “flap” in the bacterial RNA polymerase structure. The “flap loop” extending from the top of the wall, disordered in Pol II, corresponds to a loop six residues longer in E. coli that is ordered in the bacterial polymerase structure.

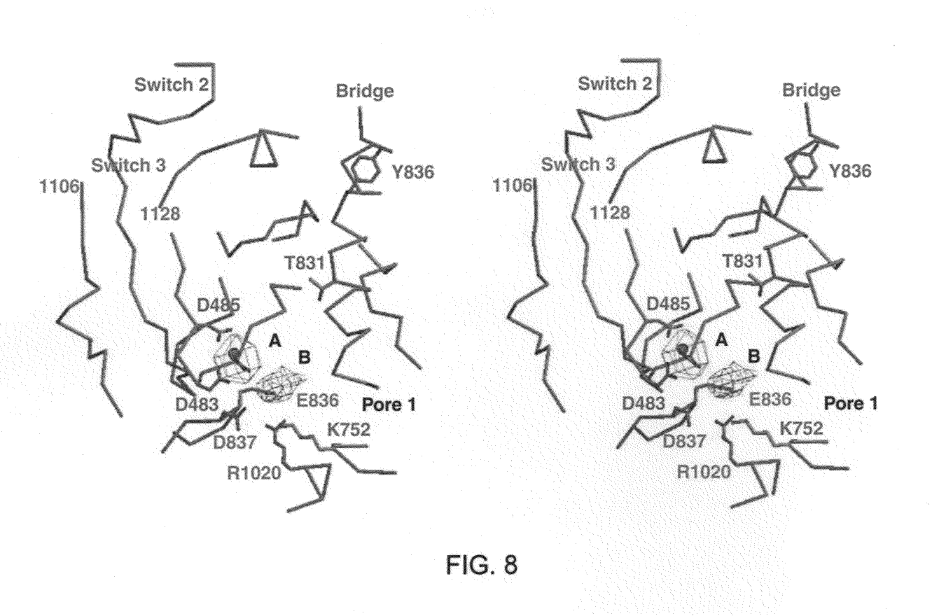

Two metal ions at the active site. A Mg²⁺ ion, bound by the invariant aspartates D481, D483, and D485 of Rpb1, identifies the active site of Pol II and is here referred to as metal A. At the corresponding position in the structure of a bacterial RNA polymerase, a metal ion was previously detected as well. The presence of only a single metal ion was unexpected, because a two-metal-ion mechanism had been proposed for all nucleic acid polymerases on the basis of x-ray studies of single-subunit enzymes. We now present evidence at the higher resolution of the form 2 data for a second metal ion in the Pol II active site. A difference Fourier map computed with only the protein structure and no metals contained two peaks, one at 21.0σ owing to metal A, and a second at 4.6σ, designated metal B (FIG. 8). Peaks with comparable relative intensities were observed at the same locations in anomalous difference Fourier maps computed for the Mn²⁺-soaked crystal. Metal B was not included in the structure because of its low occupancy.

Three observations suggest that metal B is part of the active site and that it corresponds to the second metal ion of single-subunit polymerases. (i) Metal B is in the vicinity of metal A, at a distance of 5.8 Å, compared with about 4 Å in the single-subunit polymerases. (ii) Metal B is located near three invariant acidic residues—D481 in Rpb1, and E836 and D837 in Rpb2 (FIG. 8), with aspartate D481 located between the two metals—resembling the situation in several single-subunit polymerases. The distance from metal B to the acidic residues, 3 to 4 Å, is too great for coordination, but may change during transcription (see below). (iii) The general organization of the active center resembles that of T7 RNA polymerase and DNA polymerases of various families. The two metal ions in Pol II are accessible to substrates from one side, and the Rpb1 helix bridging the cleft to Rpb2 is in about the same location relative to the metal ions as a helix in several single-subunit polymerases, generally referred to as the “O-helix.”

The location of the two metals is consistent with the geometry of substrate binding inferred from structures of a Pol II transcription elongation complex and of some single-subunit polymerases. In the single-subunit structures, metal A coordinates the 3′-OH group at the growing end of the RNA and the α-phosphate of the substrate nucleoside triphosphate, whereas metal B coordinates all three phosphate groups of the triphosphate. Both metals stabilize the transition state during phosphodiester bond formation. In Pol II, only metal A is persistently bound, at the upper edge of pore 1, whereas metal B, located further down in the pore, may enter with the substrate nucleotide. Orientation of the nucleotide by base pairing with the template may enable complete coordination of metal B, leading to phosphodiester bond formation.

Possible structural changes during translocation. A central mystery of all processive enzyme-polymer interactions is how the enzyme translocates along the polymer between catalytic steps without dissociation. Comparison of the Pol II structure with that of bacterial RNA polymerase has given unexpected insight into this aspect of the transcription mechanism. The bridge helix, highly conserved in sequence, is straight in Pol II but bent and partially unfolded in the bacterial polymerase structure. The bridge helix contacts the end of the DNA-RNA hybrid in a Pol II transcription elongation complex, and bending of the helix may be important for maintaining nucleic acid-protein interaction during translocation.

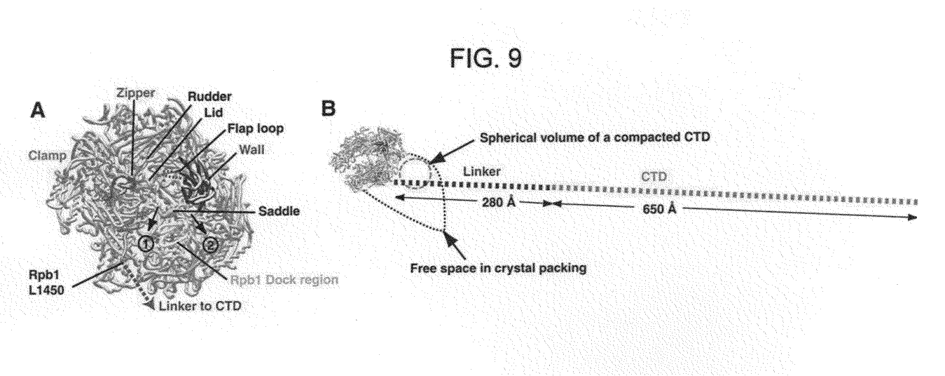

RNA exit, the CTD, and coupling of transcription to RNA processing. Two grooves in the Pol II surface were previously noted as possible paths for RNA exiting from the active-center region: “groove 1,” at the base of the clamp, and “groove 2,” passing alongside the wall (FIG. 9A). The atomic structure, together with a result from RNA-protein cross-linking, argue in favor of groove 1. A cross-link is formed to the NH₂-terminal region of β′, the homolog of Rpb1, in an E. coli transcription elongation complex. The corresponding residues in Rpb1 are located on the side of the clamp core above the beginning of groove 1 (FIG. 9A). The length of RNA in groove 1 may be short, because it enters at about residue 12 and becomes accessible to nuclease digestion at about residue 18 in Pol II and at about residue 15 in the bacterial enzyme. RNA in this part of groove 1 would lie on the saddle, beneath the Rpb1 lid and Rpb2 “flap loop.” As noted above, the surface of the saddle is positively charged, appropriate for nucleic acid interaction.

Soon after exiting from the polymerase, RNA must be available for processing, because capping occurs upon reaching a length of about 25 residues. Consistent with this requirement, the exit from groove 1 is located near the last ordered residue of Rpb1, L1450, at the beginning of the linker to the CTD (FIG. 9B), and capping and other RNA processing enzymes interact with the phosphorylated form of the CTD. It may be argued that the length of the linker would allow the CTD to reach any point on the Pol II surface (FIG. 9B), and nuclear magnetic resonance (NMR) and circular dichroism studies have demonstrated a disordered state of a free, unphosphorylated CTD-derived peptide. The absence of electron density in Pol II maps owing to the linker and CTD provides evidence of motion or disorder, but even if disordered, the linker and CTD are unlikely to be in an extended conformation. The linker and CTD regions of four neighboring Pol II molecules share a space in the crystal sufficient to accommodate them only in a compact conformation (FIG. 9B).

Whereas the 5′ end of the RNA exits through groove 1 during RNA synthesis and forward movement of Pol II, the 3′ end of the RNA is extruded during retrograde movement of the enzyme. The previous backbone model suggested extrusion through pore 1 into a “funnel” on the back side of the enzyme. Transcription factor TFIIS, which provokes cleavage of extruded RNA, was thought to bind in the funnel as well. The atomic structure of Pol II lends support to these previous suggestions. A fragment of the largest bacterial polymerase subunit that can be cross-linked to the end of extruded RNA is located in the funnel (FIG. 6). Further, Rpb1 residues that interact either physically or genetically with TFIIS cluster on the outer rim of the funnel (FIG. 6). The Gre proteins, bacterial counterparts of TFIIS, also bind to the rim of the funnel. A cluster of mutations that cause resistance to the mushroom toxin α-amanitin is located in the funnel as well (FIG. 6).

Implications for the initiation of transcription. The previous Pol II backbone model posed a problem for initiation because DNA entering the cleft and passing through the model would have to bend at the wall, whereas promoter DNA around the start site of transcription must be essentially straight (before binding to the enzyme and melting to form a transcription bubble). The only apparent solution to the problem, passage of promoter DNA over the wall, was unappealing because the DNA would be suspended over the cleft, far above the active center. A large movement of the DNA would be required for the initiation of transcription.

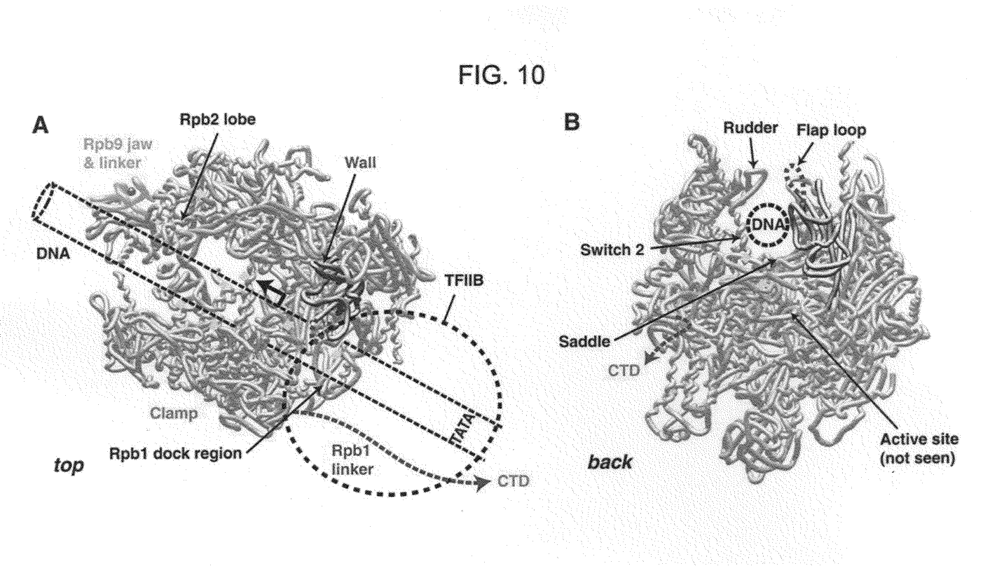

The form 2 structure suggests a new and more plausible solution of the initiation problem. In form 2, the clamp has swung further away from the active-center region, opening a wider gap than in form 1. A path is created for straight duplex DNA through the cleft from one side of the enzyme to the other (FIG. 10). The path for straight DNA is offset by 20° to 300 from the path of DNA entering a transcribing complex. Movement of DNA to this extent in the transition from an initiating to a transcribing complex seems plausible, because the DNA in this region is loosely held in the transcribing complex; the jaws, lobe, and clamp surrounding it are mobile; and a far larger movement of upstream DNA occurs upon promoter melting. Following this path, the DNA contacts the jaw domain of Rpb9, fits into a concave surface of the Rpb2 lobe, and passes over the saddle, where it is surrounded by switch 2, switch 3, the rudder, and the flap loop. These surrounding elements probably do not impede entry of DNA, because they are all poorly ordered or disordered.

Genetic evidence supports the proposed path for straight DNA during the initiation of transcription. A Pol II mutant lacking Rpb9 is defective in transcription start site selection, and complementation of the mutant with the Rpb9 jaw domain relieves the defect. Mutations in Rpb1 and Rpb2 affecting start site selection or otherwise altering initiation lie along the proposed path as well (FIG. 10). Some of these mutations are in residues that could contact the DNA, whereas others are in residues that may interact with general transcription factors.

Previous biochemical studies have suggested that the general transcription factor TFIIB bridges between the TATA box of the promoter and Pol II during initiation. Structural studies led to the suggestion that TFIIB brings a TFIID-TATA box complex to a point on the Pol II surface from which the DNA can run straight to the active center. A conserved spacing of about 25 base pairs between the TATA box and transcription start site in Pol II promoters would correspond to the straight distance to the active center. This hypothesis for transcription start site determination is consistent with the path for straight DNA proposed here. There is space appropriate for a protein the size of TFIIB between a TATA box some 25 base pairs (85 Å) from the active center and the Pol II surface (FIG. 10). TFIIB in this location would contact a region of Pol II around the Rpb1 “dock” domain that is not conserved in the bacterial polymerase sequence or structure. The proposed site of interaction with TFIIB, in the vicinity of the “dock” domain, is unrelated to a site seen previously in a difference Fourier map of a two-dimensional TFIIB-Pol II cocrystal. The difference peak attributed to TFIIB was small and may have been misleading. Binding of TFIIB in this area would also explain its interaction with an acidic region of Rpb1 that includes the adjacent “linker”.

Once bound to Pol II, promoter DNA must be melted for the initiation of transcription by the adenosine 5′-triphosphate-dependent helicase activity of general transcription factor TFIIH. The region to be melted, extending from the transcription start site about half way to the TATA box, passes close to the active center and across the saddle. As the template single strand emerges, it can bind to nearby sites in the active center, on the floor of the cleft and along the wall, where it is localized in a transcribing complex. The transition from duplex to melted promoter would thus be effected with minimal movement of protein and DNA. The transition would also remove duplex DNA from the saddle, clearing the way for RNA, whose exit path crosses the saddle.

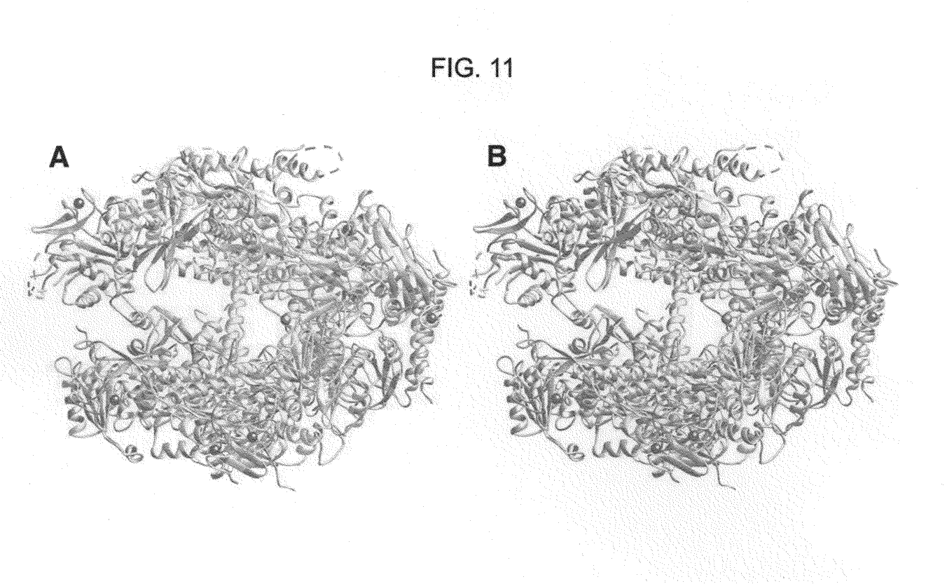

Conservation of RNA polymerase structure. All 10 subunits in the Pol II structure are identical or closely homologous to subunits of RNA polymerases I and III. Pol II is also highly conserved across species. Yeast and human Pol II sequences exhibit 53% overall identity, and the conserved residues are distributed over the entire structure (FIG. 11A). The yeast Pol II structure is therefore applicable to all eukaryotic RNA polymerases.

Some of the amino acid differences between Pol I, Pol II, and Pol III may relate to the specificity of assembly. A complex of Rpb3, Rpb10, Rpb11, and Rpb12 anchors Rpb1 and Rpb2 in Pol II and appears to direct their assembly. Rpb10 and Rpb12 are also present in Pol I and Pol III, together with homologs of Rpb3 and Rpb11, designated AC40 and AC19. Residues that interact with the common subunits Rpb10 and Rpb12 are conserved between the three polymerases. Most residues in the interface between Rpb3 and Rpb11 differ in the homologs, accounting for the specificity of heterodimer formation. Moreover, an important part of the Rpb2-Rpb3 interface (strand β10 of Rpb2 and “loop” region of Rpb3) is not conserved, which may account for the specificity of AC40 (Rpb3 homolog) interaction with the second largest subunits of Pol I and Pol III.

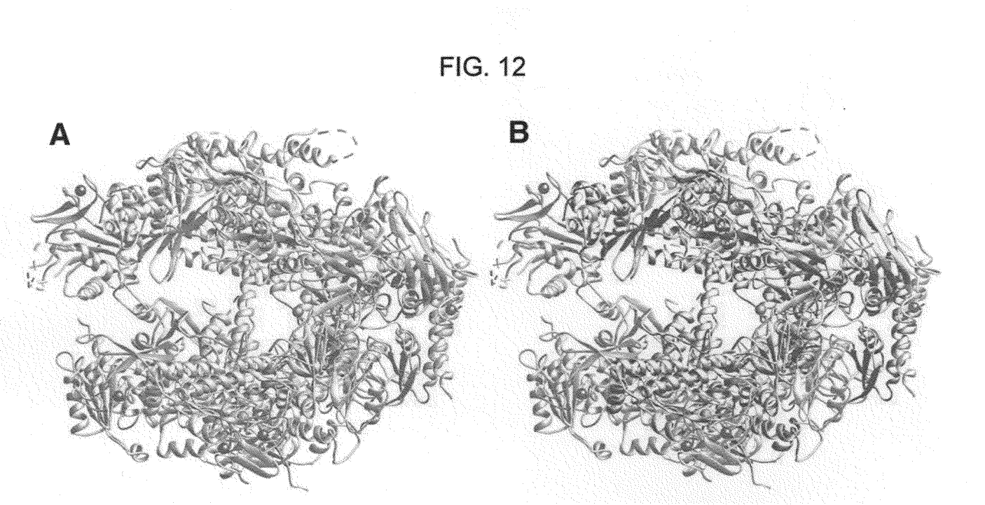

Sequence conservation between yeast and bacterial RNA polymerases is far less than for yeast and human enzymes. Identical residues are scattered throughout the structure (FIG. 11B). Regions of sequence homology between eukaryotic and bacterial RNA polymerases, however, cluster around the active center (FIG. 12A). Structural homology, determined by comparison of the Pol II protein folds with the bacterial RNA polymerase structure, is even more extensive (FIG. 12B). Yeast Pol II evidently shares a core structure, and thus a conserved catalytic mechanism, with the bacterial enzyme, but differs entirely in peripheral and surface structure, where interactions with other proteins, such as general transcription factors and regulatory factors, take place.

The immediate implications of the atomic Pol II structure are for understanding the transcription mechanism. The structure has given insight into the formation of an initiation complex, the transition to a transcribing complex, the mechanism of the catalytic step in transcription, a possible structural change accompanying the translocation step, the unwinding of RNA and rewinding of DNA, and the coupling of transcription to RNA processing. No less important are the implications for future genetic and biochemical studies of all RNA polymerases. The atomic structure provides a basis for interpretation of available data and the design of experiments to test hypotheses, such as those advanced here, for the transcription mechanism. Amino acid residues of structural elements such as the bridge helix, rudder, lid, zipper, and so forth may be altered by site-directed mutagenesis to assess their roles. Homology modeling of human RNA polymerase II will enable structure-based drug design.

### Example 2

### Structure of an Elongation Complex

The crystal structure of RNA polymerase II in the act of transcription was determined at 3.3 Å resolution. Duplex DNA is seen entering the main cleft of the enzyme and unwinding before the active site. Nine base pairs of DNA-RNA hybrid extend from the active center at nearly right angles to the entering DNA, with the 3′ end of the RNA in the nucleotide addition site. The 3′ end is positioned above a pore, through which nucleotides may enter and through which RNA may be extruded during back-tracking. The 5′-most residue of the RNA is close to the point of entry to an exit groove. Changes in protein structure between the transcribing complex and free enzyme include closure of a clamp over the DNA and RNA and ordering of a series of “switches” at the base of the clamp to create a binding site complementary to the DNA-RNA hybrid. Protein-nucleic acid contacts help explain DNA and RNA strand separation, the specificity of RNA synthesis, “abortive cycling” during transcription initiation, and RNA and DNA translocation during transcription elongation.

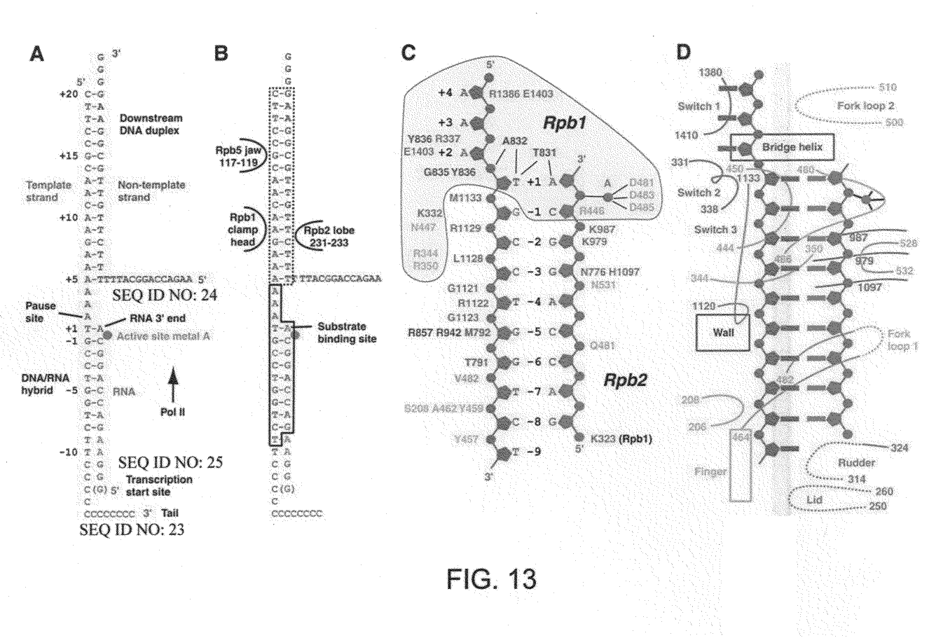

The main technical challenge of this work was the isolation and crystallization of a transcribing complex. Initiation at an RNA polymerase II promoter requires a complex set of general transcription factors and is poorly efficient in reconstituted systems. Moreover, most preparations contain many inactive polymerases, and the transcribing complexes obtained would have to be purified by mild methods to preserve their integrity. The initiation problem was overcome with the use of a DNA duplex bearing a single-stranded “tail” at one 3′-end (FIG. 13A). Pol II starts transcription in the tail, two to three nucleotides from the junction with duplex DNA, with no requirement for general transcription factors. All active polymerase molecules are converted to transcribing complexes, which pause at a specific site when one of the four nucleoside triphosphates is withheld. The problem of contamination by inactive polymerases was solved by passage through a heparin column; inactive molecules were adsorbed, whereas transcribing complexes flowed through, presumably because heparin binds in the positively charged cleft of the enzyme, which is occupied by DNA and RNA in transcribing complexes. The purified complexes formed crystals diffracting anisotropically to 3.1 Å resolution.

Plate-like monoclinic crystals of space group C2 with unit cell dimensions a=157.3 Å, b=220.7 Å, c=191.3 Å, and β=97.5° were grown by the sitting drop vapor diffusion method under the conditions previously developed for free pol II (Fu et al., (1999) Cell 98, 799). Crystals were transferred slowly to freezing buffer and flash frozen in liquid nitrogen. Diffraction data were collected at a wavelength of 0.998 Å at beamline 9.2 at the Stanford Synchrotron Radiation Laboratory. Although diffraction to 3.1 Å resolution could be observed in two directions, anisotropy limited the useable data to 3.3 Å resolution.

Structure of a pol II transcribing complex. Diffraction data complete to 3.3 Å resolution were used for structure determination by molecular replacement with the 2.8 Å pol II structure. Data processing with DENZO and SCALEPACK (Otwinowski and Minor (1996) Methods Enzymol. 276, 307) showed that the data collected at 0.998 Å were 100% complete in the resolution range 40 to 3.3 Å. A total of 96,867 unique reflections were measured. At a redundancy of 4.4, the Rsym was 11.1% (31.7% at 3.4 to 3.3 Å). The structure was solved by molecular replacement with AMORE [Navaza (1994) Acta Crystallogr. A50, 157). A modified atomic pol II structure lacking the mobile clamp was used as search model. A single strong peak was obtained after rotation and translation searches (correlation coefficient=59, R factor=43%, 15 to 6.0 Å resolution).

A native zinc anomalous difference Fourier map showed peaks coinciding with five of the eight zinc ions of the pol II structure, confirming the molecular replacement solution. Diffraction data were recollected at the zinc anomalous peak wavelength (1.283 Å) from the crystal used in structure determination. Initial phases were calculated from the pol II search model after rigid body refinement in CNS.

The remaining three zinc ions were located in the clamp, a region shown previously to undergo a large conformational change between different pol II crystal forms. The locations of the three zinc ions served as a guide for manual repositioning of the clamp in the transcribing complex structure. An initial electron density map revealed nucleic acids in the vicinity of the active center. After adjustment of the protein model, the nucleic acid density improved and nine base pairs of DNA-RNA hybrid could be built. Model building was carried out with the program O (Jones et al. (1991) Acta Crystallogr. A 47, 110) and refinement was carried out with CNS. For cross validation, 10% of the data were excluded from refinement. The four mobile modules defined for free pol II were used for rigid body refinement, followed by bulk solvent correction and anisotropic scaling. After positional and restrained B-factor refinement, a free R-factor of 35% was obtained with all data. The resulting sigma-weighted electron density maps allowed building of switch 3 and rebuilding of the other switch regions. Loops that were present in free pol II but disordered in the transcribing complex were removed. The final protein electron density was generally of good quality and most side chains were visible. Some flexible regions, including the jaws, parts of Rpb8, and the upper portions of the wall and clamp, showed only main chain density. In these regions, the refined pol II structure was not rebuilt. A few rounds of model building and refinement of the protein lowered the free R factor to 31.0%. At this stage, difference density with a helical shape was observed for the nucleic acids in the hybrid region and phosphates and bases were revealed. The density originating at the active site metal was assigned to the RNA strand, and the opposite continuous density was assigned to the DNA template strand. A total of 22 nucleotides were placed individually, resulting in a 0.7% drop in the free R factor after refinement.

Additional density along the DNA template strand allowed another three nucleotides downstream and one nucleotide upstream to be built. Modeling of the nucleic acids assumed the 3′-end of the RNA at the biochemically defined pause site (FIG. 13A), because the nucleic acid sequences could not be inferred from the crystallographic data. The 3.3 Å electron density map did not allow distinction of purine from pyrimidine bases. Placement of the particular sequences thus assumed complete RNA synthesis until the pause site and no back-tracking. Modeling resulted in a length of the downstream DNA that agrees with end-to-end packing of DNAs from neighboring complexes. The ambiguity in the assignment of nucleic acid sequences does not affect the conclusions because there are no base-specific protein contacts. The density map included a few weak, disconnected peaks in pore 1 that may arise from back-tracked RNA in a subpopulation of complexes or from incoming nucleoside triphosphates.

The final model contains 3521 amino acid residues, 22 nucleotides, eight Zn²⁺ ions, and one Mg²⁺ ion and has a free R factor of 29.8% (R factor 25.0%, 40 to 3.3 Å) (FIG. 14). A simulated-annealing omit map computed from a model of the protein alone revealed the phosphate groups and most bases in the DNA-RNA hybrid region, confirming the modeling of the nucleic acids (FIG. 14A). Density for DNA in the downstream region was very weak and discontinuous but revealed the major groove, allowing a canonical B-DNA duplex to be approximately placed. At the standard contour level of 1.0, only a few disconnected peaks are observed for the downstream DNA. At a contour level of 0.8, extended density features are observed, which identify the approximate helix axis and major groove of the downstream DNA, with only a few disconnected noise peaks in the surrounding solvent region. Inclusion of the DNA duplex placed in this way in the refinement led to an increase in the free R factor. Numbering of nucleotides in the DNA begins with +1 immediately downstream and −1 upstream of the Mg²⁺ ion (FIG. 13A).

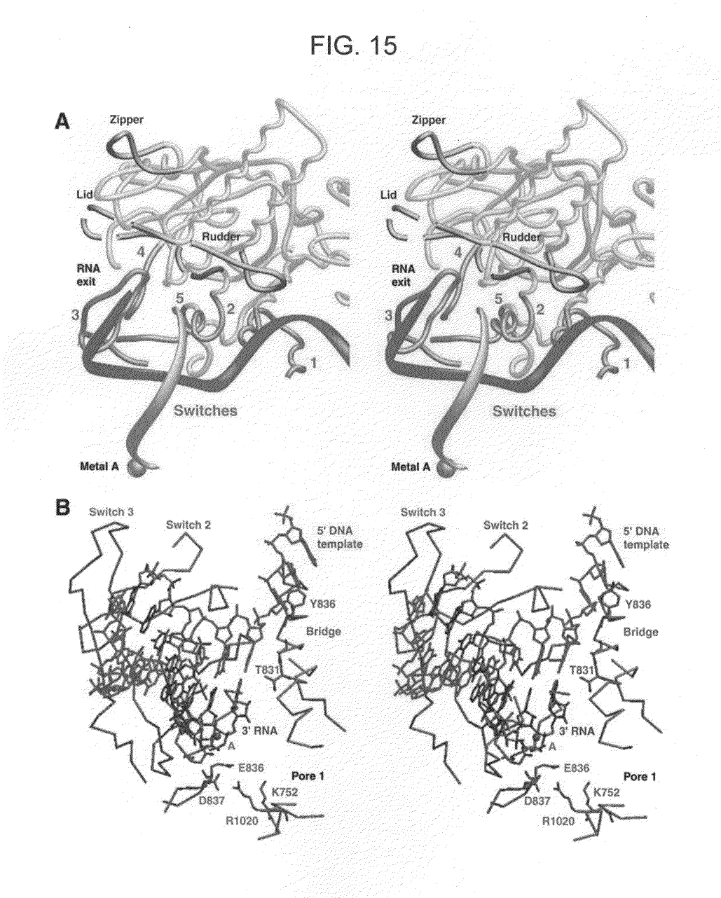

Closure of the clamp. The structures of free and transcribing pol II differ mainly in the position of the clamp (FIG. 14B). The clamp swings over the cleft during formation of the transcribing complex, trapping the template and transcript. The clamp rotates by about 30°, with a maximum displacement of over 30 Å at external sites (at the Rpb1 “zipper”). Although most of the clamp moves as a rigid body, five “switch” regions undergo conformational changes and folding transitions (Table 5). Switches 1, 2, 4, and 5 form the base of the clamp (FIG. 15). Switches 1 and 2 are poorly ordered and switch 3 is disordered in free pol II; all three switches become well ordered in the transcribing complex. Ordering is likely induced by binding of the switches to DNA downstream and within the DNA-RNA hybrid. Binding to the hybrid may help couple clamp closure to the presence of RNA. The conformational changes of the switch regions may be concerted, because the switches interact with one another. The conformational changes are accompanied by changes in a network of salt linkages to the “bridge” helix across the cleft (Rpb1 residues Arg⁸³⁹, Arg⁸⁴⁰, and Lys¹⁴³).

TABLE 5
Switch regions.

| | | | | DNA | Structural changes |
|---|---|---|---|---|---|
| Switch | Subunit | Domain | Residues | contact | upon clamp closure |
| 1 | Rpb1 | Cleft-clamp core | 1384 1406 | +1 to +4 | Two short helices |
| | | | | | formed (47a, 47b) |
| 2 | Rpb1 | Clamp core | 328 346 | 2, 1, +2 | Helical turn flipped out |
| 3 | Rpb2 | Hybrid-binding | 1107 1129 | 5 to 1 | Loop becomes |
| | | anchor | | | ordered |
| 4 | Rpb2 | Clamp | 1152 1159 | — | One turn added to |
| | | | | | helix. 32 in the |
| | | | | | anchor region |
| 5 | Rpb1 | Clamp core | 1431 1433 | — | Hinge-like bending |

Downstream DNA mobility. Downstream DNA lies in the cleft between the clamp and Rpb2 (FIGS. 13B and 14B and C), consistent with results from electron crystallography of the transcribing complex and results of DNA-protein cross linking. The DNA contacts the Rpb5 “jaw” domain at a loop containing proline residue Pro¹¹⁸, and then passes between the Rpb2 “lobe” region and the Rpb1 “clamp head.” The sequence of the Rpb2 lobe is divergent between yeast and bacteria, but the fold is conserved, whereas the clamp head is not conserved.

Details of downstream DNA-pol II interaction are lacking because the electron density is weak, indicative of mobility of the DNA. Furthermore, downstream DNAs from neighboring transcribing complexes in the crystal interact end to end, stacking on one another, so the precise location of the DNA may be determined by crystal packing forces. This could be the reason why there is no apparent contact between downstream DNA and the upper jaw. In addition, the length of DNA used here is possibly too short for passage all the way through the jaws.

Transcription bubble. The downstream edge of the transcription bubble lies between the poorly ordered downstream duplex DNA and the first ordered nucleotide of the template strand at position +4, three nucleotides before the beginning of the RNA-DNA hybrid (FIG. 15B). The nucleotide at position +4 in the nontemplate strand and the remainder of this strand are disordered. The template strand follows a path along the bottom of the clamp and over the “bridge” helix. Template nucleotides +4, +3, and +2 are stacked in the manner of right-handed B-DNA. The base of nucleotide +1 is flipped with respect to that of nucleotide +2 by a left-handed twist of 900. The base at +1 therefore points downward into the floor of the cleft for readout at the active site, whereas the base at +2 is directed upward into the opening of the cleft. This unusual conformation of the DNA results from binding to switches 1 and 2, as well as to the bridge helix (FIGS. 13C and D). Invariant bridge helix residues Ala⁸³² and Thr⁸³¹ position the coding nucleotide through van der Waals interactions, whereas Tyr⁸³⁶ binds nucleotide +2 and may correspond to a tyrosine in the “O-helix” of some single subunit DNA polymerases.

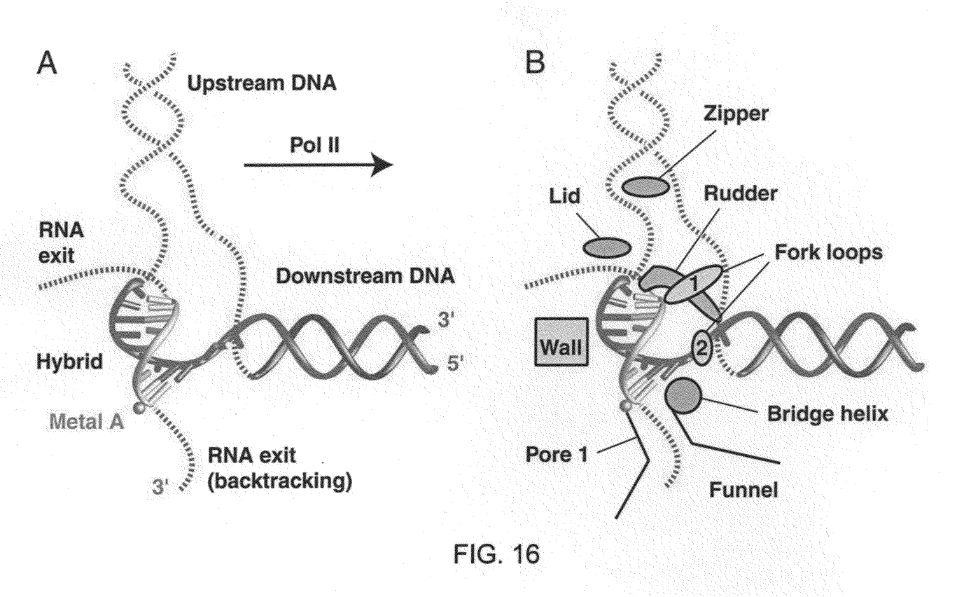

Maintenance of the downstream edge of the transcription bubble may be attributed not only to the binding of nucleotides +2, +3, and +4 but also to Rpb2 “fork loop” 2 (FIG. 13D and FIG. 16). Although this loop includes several disordered residues, it would likely clash with the nontemplate strand at position +3 if the nontemplate strand was still base paired with the template strand. A corresponding loop in the bacterial enzyme (“βD loop I”), four residues longer than that in yeast, was previously suggested to play such a role. Rpb2 fork loop 1 may help maintain the transcription bubble further upstream (FIG. 13D and FIG. 16). This loop is absent from the bacterial enzyme, perhaps reflecting a difference in promoter melting between eukaryotes, which require general transcription factors for the process, and bacteria, which do not. Both fork loops, although exposed, are highly conserved between yeast and human polymerases.

DNA-RNA hybrid. The base in the template strand at position +1 forms the first of nine base pairs of DNA-RNA hybrid, located between the bridge helix and Rpb2 “wall” (FIG. 13D and FIG. 16). The length of the hybrid corroborates the value of eight to nine base pairs determined biochemically. The hybrid heteroduplex adopts a nonstandard conformation, intermediate between those of standard A- and B-DNA (FIG. 17), and is underwound, in comparison with the crystal structure of a free DNA-RNA hybrid, which is closely related to the A-form.

The nucleic acid model was obtained by placing nucleotides manually into unbiased electron density peaks. At 3.3 Å resolution, the location of phosphate groups and the approximate axes through base pairs were revealed. After refinement, the positions of the nucleotides changed only slightly, showing that the final nucleic acid model reflects the experimental data and that the model is not primarily a result of the geometrical constraints applied during refinement. Although the available data define the overall hybrid conformation, stereochemical details are not revealed and the parameters of the hybrid helix must be viewed as approximate. The hybrid shows an average rise per residue of 3.2 Å {program CURVES (Layery and Sklenar (1988) J. Biomol. Struct. Dyn. 6, 63), compared with 2.8 and 3.4 Å for A- and B-DNA, respectively. The average minor groove width is 10.4 Å (CURVES), compared with 11 and 7.4 Å for A- and B-DNA, respectively. The root-mean-square (rms) deviation in phosphorus atom positions between the hybrid and canonical A- and B-DNA is 3.1 and 5.5 Å, respectively. The helical twist is 12.6 residues/turn {program NEWHELIX (Grzeskowiak et al. (1993) Biochemistry 32, 8923). The phosphorus atom positions show an rms deviation of 2.7 Å from the structure of a free hybrid.

The electron density for the hybrid is strongest in the downstream region around the active center, indicative of a high degree of order, important for the high fidelity of transcription. The electron density remains strong for the DNA template strand further upstream, but the density for the RNA strand becomes weaker (FIG. 14A). This gradual loss of density reflects a diminution in the number of RNA-protein contacts. The template DNA strand is bound by protein over the entire length of the hybrid, whereas RNA contacts are limited to the downstream region (FIG. 13C). The five upstream ribonucleotides are held mainly through base pairing with the template DNA.

Contacts to the downstream and upstream parts of the hybrid are made by Rpb1 and Rpb2, respectively (FIG. 1C). Fifteen protein regions are involved, with a substantial portion of the contacts arising from the ordering of Rpb1 switches 1, 2, and 3 upon nucleic acid binding. The entire set of protein contacts forms an extended, highly complementary binding surface. A surface area of 3400 A² is buried in the protein-nucleic acid interface, comparable to values for transcription factors bound specifically to DNA sites of similar size. Biochemical studies have shown the binding interaction contributes substantially to the stability of a transcribing complex and thus to the high processivity of transcription.

Although a strong pol II-nucleic acid interaction is important for the ordering of nucleic acids in the active center region and for the stability of a transcribing complex, the interaction must not interfere with the translocation of nucleic acids during transcription. Indeed, the nucleic acids in the transcribing complex are mobile, as shown by the partial order of the downstream DNA and by a high overall crystallographic temperature factor of the hybrid, which appears to reflect mobility rather than static disorder. The average atomic B factor is 97 A2 for the hybrid, as compared with 63 Å2 for the entire structure. The bases and backbone groups show similar B factors. This likely indicates mobility because static disorder, arising from the presence of complexes at different register, would be expected to result in low B factors for the backbone and higher B factors for the bases. Refinement of atomic B factors is justified at the given resolution and that the resulting B factors are meaningful, because refinement of all protein atoms, starting from a constant value of 30 Å2, results in an overall B factor that is very close to that obtained for the free pol II structure at 2.8 Å resolution. Moreover, the general distribution of B factors is similar to that for the structure of free pol II.

The conflicting requirements of tight binding and mobility may be reconciled in at least three ways. First, almost all protein contacts are to the sugar-phosphate backbones of the DNA and RNA. There are no contacts with the edges of the bases, so there is no base specificity. A large open space between pol II and the major groove of the hybrid is a prominent feature of the structure. Second, several side chains interact with two phosphate groups along the backbone simultaneously (FIG. 13C), which may reduce the activation barrier for translocation. Finally, about 20 positively charged side chains form a “second shell” around the hybrid at a distance of 4 to 8 Å, which may attract the hybrid without restraining its movement across the enzyme surface. These residues include arginines 320, 326, 839, and 840 and lysines 317, 323, 330, 343, and 830 of Rpb1 and arginines 476, 497, 766, 1020, 1096, and 1124 and lysines 210, 458, 507, 775, 865, 965, and 1102 of Rpb2.

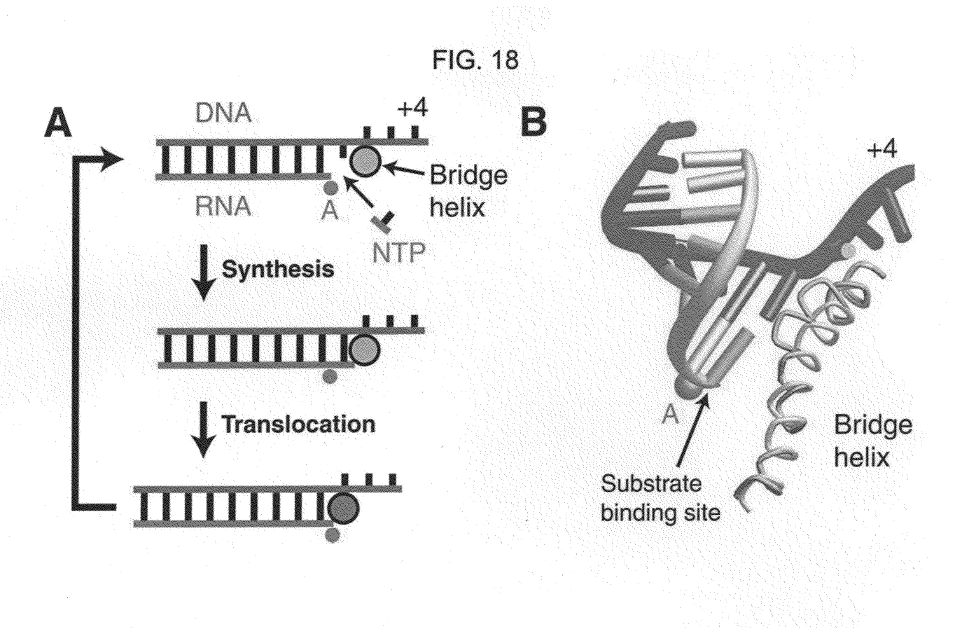

RNA synthesis. The active site metal ion in the transcribing complex structure corresponds to one of two metal ions in the 2.8 Å pol II structure, referred to as metal A. The location of this metal in the transcribing complex is appropriate for binding the phosphate group between the nucleotide at the 3′-end of the RNA and the adjacent nucleotide, designated +1 and −1, respectively (FIG. 13C). In the two-metal-ion mechanism proposed for single subunit polymerases, metal A contacts the α-phosphate of the incoming nucleoside triphosphate and metal B binds all three phosphates. Metal B may be absent from the transcribing complex structure because it has left with the pyrophosphate after nucleotide addition. On this basis, position +1 in the transcribing complex would be that of a nucleotide just added to the growing RNA, before translocation to bring the next template base into position opposite an empty nucleotide-binding site at the end of the RNA (FIG. 18). Although the 3′-most residue of the RNA is in the position of a nucleotide just added to the chain, it must have undergone translocation and then returned to this position before crystallization. Translocation is necessary to create a site for the next nucleotide, whose absence from the reaction results in a paused complex.

The ribonucleotide in position +1 lies in the entrance to the previously noted “pore 1,” which extends from the floor of the cleft through to the backside of the enzyme. This location and orientation of the 3′-end of the RNA lend strong support to the previous proposal that nucleoside triphosphates enter through the pore during RNA synthesis and that RNA is extruded through the pore during back-tracking. The close fit of the DNA-RNA hybrid to the surrounding protein leaves no alternative to the pore for access of nucleotides to the active site. (Major conformational changes creating access are unlikely, because they would disrupt protein-nucleic acid contacts important for the fidelity and processivity of transcription.)

Specificity for ribo—rather than deoxyribonucleotides may be attributed to recognition of both the ribose sugar and the DNA-RNA hybrid helix. The 2′-hydroxyl group of a ribonucleotide in the substrate binding site (position +1) is 5 Å from the side chain of the highly conserved Rpb1 residue Asn⁴⁷⁹. Although this distance is too great for specific interaction, a slightly different positioning of an incoming nucleoside triphosphate might permit hydrogen bonding and discrimination of the ribose sugar. Different positioning of the nucleoside triphosphate could result from chelation by metal B, bound at a site in the structure of free pol II. RNA 2′-hydroxyl groups at positions −1, −3, and −5 are at hydrogen bonding distance from the side chains of Rpb1 residue Arg⁴⁴⁶ and Rpb2 residues His¹⁰⁹⁷ and Gln⁴⁸¹. The nucleic acid binding site is, furthermore, highly complementary to the nonstandard conformation of the hybrid helix and not to the standard conformation of a DNA double helix. Such indirect discrimination was previously suggested to contribute to the specificity of T7 RNA polymerase transcription.

Recognition of RNA in the transcribing complex from positions −1 to −5, by both hydrogen bonding and indirect discrimination, can contribute to the specificity of RNA synthesis through proofreading. The presence of a deoxyribonucleotide or of an incorrect base anywhere in this region of the RNA will be destabilizing. A back-tracked complex, with previously correctly synthesized RNA in the hybrid region and with the RNA containing the misincorporated nucleotide extruded at the 3′-end, will be favored. The extruded RNA can be removed by cleavage at the active site, through the action of transcription factor TFIIS.

Key nonspecific (van der Waals) contacts to the nucleotide base at the end of the hybrid region, in position +1, are made by residues Thr⁸³¹ and Ala⁸³² from the Rpb1 bridge helix, as mentioned above. Although highly conserved, the bridge helix is essentially straight in the pol II structures so far determined but bent in the bacterial enzyme structure in the vicinity of the residues corresponding to Thr⁸³¹ and Ala⁸³². The bend would produce a movement of this region of the bridge helix by 3 to 4 Å, resulting in a clash with the nucleotide at position +1 (FIG. 18). Modeling of a bacterial transcribing complex resulted in such a clash. We speculate that the bridge helix oscillates between straight and bent states and that this movement accompanies the translocation of nucleic acids during transcription: Addition of a nucleotide at position +1 would occur in the straight state; translocation to position −1 and movement of nucleic acids through the distance between base pairs, about 3.2 Å, would be accompanied by a conformational change to the bent state; and reversion to the straight state without movement of nucleic acids would create an empty site at position +1 for entry of the next nucleotide, completing a cycle of nucleotide addition during RNA synthesis (FIG. 18).

Protein-RNA contacts are of special importance at the very beginning of transcription. Nucleoside triphosphates must be held in positions +1 and −1 for the synthesis of the first phosphodiester bond. After translocation to positions −1 and −2, the dinucleotide product must still be held by protein-RNA contacts, as the energy of base-pairing alone is insufficient for retention in the complex. Indeed, RNA is deeply buried in the transcribing complex as far as position −3 (FIG. 13C). Di- and trinucleotides are nevertheless occasionally released, and transcription must restart, resulting in “abortive cycling”. RNA is exposed at position −4 and beyond, with no direct protein contacts except for the hydrogen bond at position −5 mentioned above. Coincident with exposure of the RNA, biochemical studies reveal a transition in stability at a transcript length of four residues, beyond which the RNA is generally retained. Although the direct protein-RNA contacts observed up to this point may be largely responsible for retention, long-range interactions also play a role. For example, a highly conserved arginine makes long-range electrostatic interactions with the RNA around position −4 (Arg⁴⁹⁷ in Rpb2, Arg⁵²⁹ in Escherichia coli β), and mutation of this residue results in the overproduction of abortive transcripts.

RNA exit. Abortive cycling yields an abundance of two- to three-residue transcripts, as well as transcripts of up to 10 residues. An initiating complex evidently undergoes a second transition when the transcript reaches 10 residues in length. At this point, the newly synthesized RNA must separate from the DNA-RNA hybrid and enter an exit channel on the surface of the enzyme, where it remains protected from nuclease attack for about six more residues. Three loops extending from the clamp, termed “rudder,” “lid,” and “zipper,” have been suggested to play roles in hybrid dissociation, RNA exit, and maintenance of the upstream end of the transcription bubble (FIG. 16). Modeling of the DNA-RNA hybrid beyond the nine base pairs seen in the transcribing complex structure would produce a clash with the rudder. Extension of the RNA from the last hybrid base pair leads beneath the rudder to the previously proposed “exit groove 1.” Continuation of this RNA path also leads beneath the lid, whose role may be to maintain the separation of RNA and template DNA strands. The zipper may play a similar role in separating template and nontemplate DNA strands. The lid and a small portion of the rudder are disordered in the transcribing complex structure but are ordered in the free pol II structure. The lid and rudder may become ordered in the transcribing complex in conjunction with the second transition and with the establishment of a stable, elongating complex. Ordering of the rudder and lid may not be observed because of structural heterogeneity of the transcribing complexes in this region. Heterogeneity might be expected as a consequence of inefficient displacement of RNA from DNA-RNA hybrid during transcription of tailed templates.

The atomic structure of RNA polymerase II in the act of transcription reveals the protein-DNA and -RNA interactions underlying the process. The structure shows a right angle bend of the DNA path at the active center. This feature is understandable in retrospect. The bend orients the DNA-RNA hybrid optimally for transcription, which occurs along the direction of the hybrid axis. Nucleotides enter through the funnel and pore, add to the RNA at the end of the RNA-DNA hybrid, translocate through the hybrid-binding region, and exit beneath the rudder and lid.

Answers to many long-standing questions about the transcription mechanism may be found in the structure of the clamp. This mobile, multifunctional element does more than close over the nucleic acids in the active center to enhance the processivity of transcription. First, switch regions at the base of the clamp couple its closure to the presence of DNA-RNA hybrid in the active center. This coupling satisfies the dual requirement for retention of nucleic acids during transcript elongation and their release after termination. Second, through the rudder, lid, and zipper, the clamp plays a key role in the events of hybrid melting and template reannealing at the upstream end of the transcription bubble.

Testing of the roles for these structural elements by site-directed mutagenesis can now be designed on the basis of the structure. In addition, polymerase may be cocrystallized with synthetic transcription bubbles and other forms of RNA and DNA.

### Example 3

### Complex of RNA Polymerase II with an Inhibitor

The structure of 10-subunit 0.5-MDa yeast RNA polymerase II (pol II), recently determined at 2.8 Å resolution, reveals the architecture and key functional elements of the enzyme. The two largest subunits, Rpb1 and Rpb2, lie at the center, on either side of a nucleic acid-binding cleft, with the many smaller subunits arrayed around the outside. Rpb1 and Rpb2 interact extensively in the region of the active site and also through a domain of Rpb1 that lies on the Rpb2 side of the cleft, connected to the body of Rpb1 by an α-helix that bridges across the cleft.

Proof that nucleic acids bind in the channel comes from the molecular replacement solution of a transcribing pol II complex at 3.3 Å resolution. This structure shows the template DNA unwinding some three residues before the active site, followed by nine base pairs of DNA-RNA hybrid. Adjacent regions of Rpb1 and Rpb2 form a highly complementary surface, resulting in extensive DNA-RNA hybrid-protein interaction. The “bridge” helix seems to play an important role, binding to both the second and third unpaired DNA bases and also to the coding base, paired with the first residue of the RNA. Comparison of the pol II structure in different crystal forms shows a division of the enzyme in several mobile elements that my facilitate DNA and RNA movement during transcription. Comparison of the pol II structure with that of the related bacterial RNA polymerase suggests mobility of the bridge helix as well.

The pol II structures open the way to many lines of investigation. Structures of cocrystals of pol II with interacting molecules can be solved, the full power of site-directed mutagenesis can be brought to bear on the transcription mechanism, and so forth. Here we report the structure of a cocrystal of pol II with the most potent and specific known inhibitor of the enzyme, α-amanitin. The active principle of the “death cap” mushroom, α-amanitin blocks both transcription initiation and elongation. The structure of the cocrystal suggests that α-amanitin interferes with a protein conformational change underlying the transcription mechanism.

### Materials and Methods

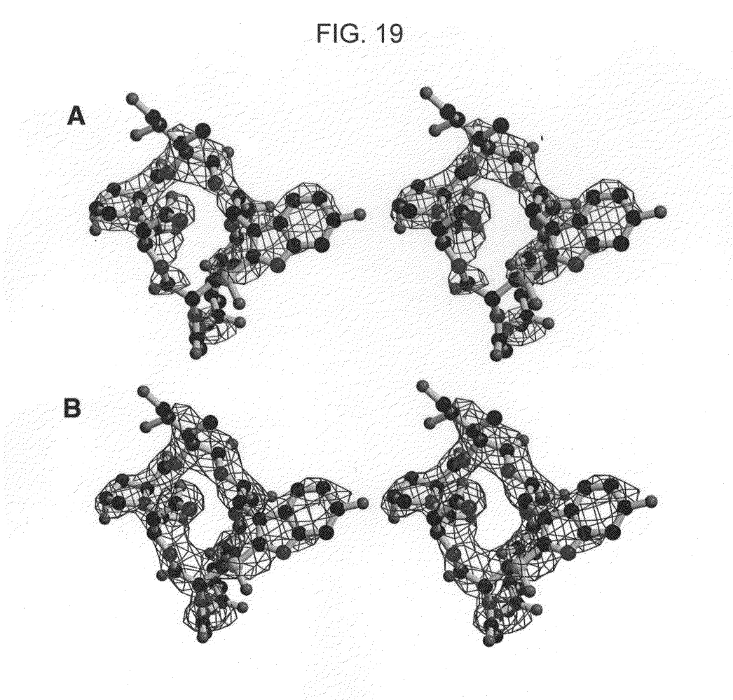

Crystals of yeast pol II were grown as described and were soaked in cryoprotectant solution containing 50 μg/ml α-amanitin and 1 mM MgSO₄ for 1 week before freezing and x-ray data collection to 2.8 Å resolution (Table 6). Data collection was carried out at 100 K by using 0.5° oscillations with an Area Detector Systems Quantum 4 charge-coupled device (CCD) detector at Stanford Synchrotron Radiation Laboratory beamline 11-1. Diffraction data were processed with DENZO and reduced with SCALEPACK. The previous 2.8-Å pol II structure was subjected to rigid body refinement against the cocrystal data. The R-free test set from the native form 2 pol II data was used for the pol II α-amanitin refinement. Refinement of the cocrystal structure was preformed by using CNS. A σA-weighted difference electron density map was consistent with the known structure of amanitin toxins (FIG. 19A). After positional and B-factor refinement of the pol II model and minor adjustments to the model, an α-amanitin model was placed. The α-amanitin model was generated from 6′-O-methyl-α-amanitin (S)-sulfoxide methanol solvate monohydrate as obtained from the Cambridge Structure Database [accession code 3384082]. To conform to the known composition and stereochemistry of α-amanitin, the 6′-O-methyl group was removed from the 6′-O-methyltryptophan residue (α-amanitin position 4) and the stereochemistry of the sulfoxide was modified to R. Topology and refinement parameter files for use in CNS for the -amanitin structure were generated by using HIC-UP. Rigid body refinement was performed on the α-amanitin alone, followed by positional and B-factor refinement of the entire pol II-α-amanitin complex and further minor adjustment of the model, giving a final free-R factor of 28% (Table 7). The refined σA-weighted 2F_{obs}−F_{calc} map (FIG. 19B) clearly shows density for the main chain atoms. Some of the side chains, however, such as that of the 4,5-dihydroxyisoleucine residue, are only partially visible (ordered) in the map. The stereo chemistry of the 4,5-dihydroxyisoleucine γ hydroxyl is important in amanitin inhibition, suggestive of a role in hydrogen bonding. Poor ordering in our cocrystal indicates that at least in yeast, the proposed hydrogen bond is not formed. This may partially explain the lesser sensitivity of Saccharomyces cerevisiae to α-amanitin compared with other eukaryotes.

TABLE 6
Crystallographic data

| Space group | I222 |
|---|---|
| Unit cell, Å | 122.5 by 222.5 by 374.2 |
| Wavelength, Å | 0.965 |
| Mosaicity, ° | 0.44 |
| Resolution, Å | 20-2.8 (2.9-2.8) |
| Completeness, % | 99.8 (99.4) |
| Redundancy | 3.9 (2.9) |
| Unique reflections | 124,441 (12,292) |
| R_{sym}, % | 6.7 (21.6) |

### Results and Discussion

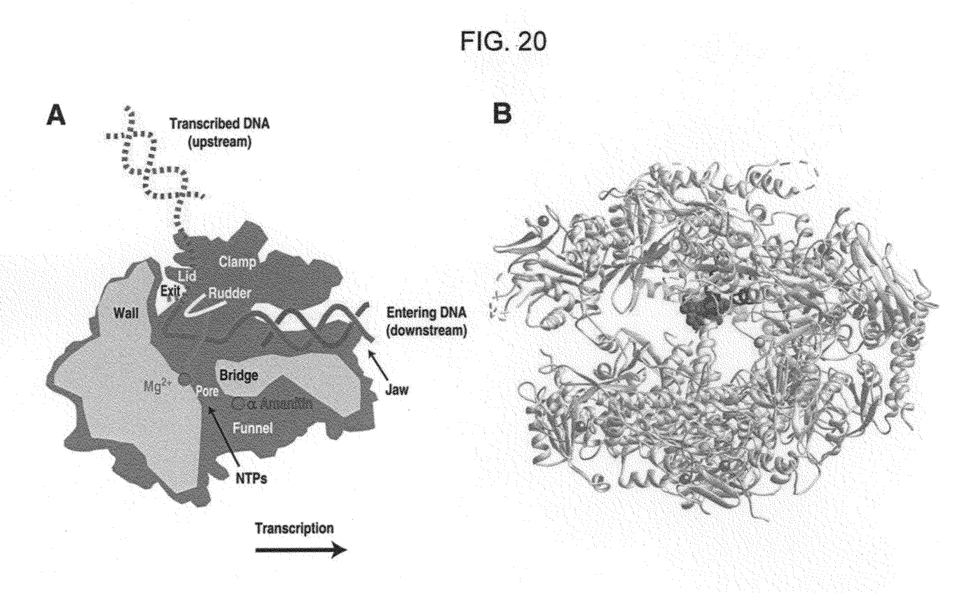

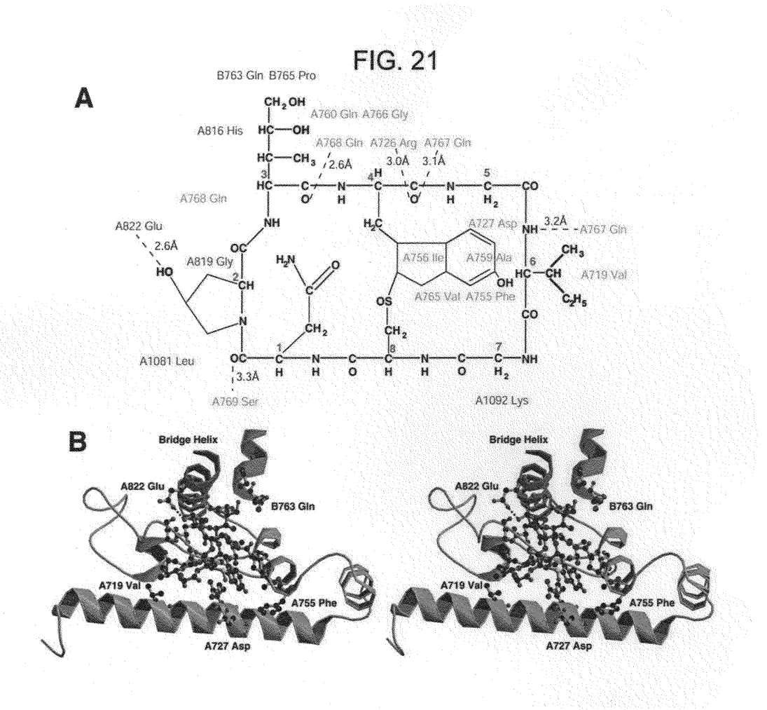

The α-amanitin binding site is beneath a “bridge helix” extending across the cleft between the two largest pol II subunits, Rpb1 and Rpb2, in a “funnel”-shaped cavity in the pol II structure (FIGS. 20A and B). Most pol II mutations affecting α-amanitin inhibition map to this site (Table 7), showing that it is functionally relevant and not an artifact of crystallization. Pol II residues interacting with α-amanitin are located almost entirely in the bridge helix (in the previously defined “cleft” region of Rpb1) and in an adjacent part of Rpb1 on the Rpb2-side of the cleft [in the previously defined funnel region of Rpb1 (FIGS. 21A and B; Table 7)]. There is a strong hydrogen bond between hydroxyproline 2 of α-amanitin and bridge helix residue Glu-A822. There is an indirect interaction involving the backbone carbonyl group of 4,5-dihydroxyisoleucine 3 of α-amanitin, hydrogen-bonded to residue Gln-A768, which is, in turn, hydrogen-bonded to bridge helix residue His-A816. Finally, there are several hydrogen bonds between α-amanitin and the region of Rpb1 adjacent to the bridge helix. Binding of α-amanitin therefore buttresses the bridge helix, constraining its position with respect to the Rpb2-side of the cleft.

TABLE 7
Refinement statistics

| | |
|---|---|
| Nonhydrogen atoms | 27,906 |
| Protein residues | 3,490 |
| Water molecules | 69 |
| Anisotropic scaling (B11, B22, B33) | −6.3, −6.9, 13.1 |
| rms deviation bonds | 0.0083 |
| rms deviation angles | 1.4 |
| Reflection test set | 3,757 (3.0%) |
| R_{cryst}/R_{free} | 22.9/28.0 |
| Average B factor overall | 57 |
| Average B factor pol | 57 |
| Average B factor amanitin | 78 |
| Average B factor water | 35 |

R_{cryst/free} = Σₕ || F_{obs}(h)| − |F_{calc}(h) || /Σₕ|F_{obs}(h)|. R_{cryst} and R_{free} were calculated from the working and test reflection sets, respectively.

This mode of α-amanitin interaction can account for the biochemistry of inhibition. There is little if any influence of α-amanitin binding on the affinity of pol II for nucleoside triphosphates. Moreover, after the addition of α-amanitin to a transcribing pol II complex, a phosphodiester bond can still be formed. The rate of translocation of pol II on DNA is, however, reduced from several thousand to only a few nucleotides per minute. These findings are consistent with binding of α-amanitin too far from the active site to interfere with nucleoside triphosphate entry or RNA synthesis (or its reversal) (FIG. 20A). They may be explained by a constraint on bridge helix movement. It was previously suggested that such movement is coupled to DNA translocation. The suggestion was based on two observations. First, in the structure of a pol II-transcribing complex, bridge helix residues directly contact the DNA base paired with the first base in the RNA strand. Second, although the sequence of the bridge helix is well conserved, the conformation is different in a bacterial RNA polymerase structure, with bridge helix residues in position to contact the second base in the DNA strand. Movement of bridge helix residue Glu-A822 by as little as 1 Å would extend the length of the donor-acceptor pair for the hydrogen bond to hydroxyproline 2 of α-amanitin beyond 3.3 Å, effectively breaking the bond.

TABLE 8
Hydrogen bonds, buried surface area, and known amanitin mutants

| Residue in | Δsurface | | Residue in | |
|---|---|---|---|---|
| yeast | area, Å² | H-bond | human | Mutations |
| Val-A719 | −32 | | Asn-A742 | |
| Leu-A722 | 0 | | Leu-A745 | Mouse L745F (13) |
| Asn-A723 | −22 | | Asn-A746 | |
| Arg-A726 | −63 | NH1 to AMA | Arg-A749 | Mouse R749P (14) Drosophila |
| | | pos. 4 O 3.0 Å | | melanogaster R741H(15) |
| Asp-A727 | −7 | | Asp-A750 | |
| Phe-A755 | −8 | | Lys-A778 | |
| Ile-A756 | −48 | | Ile-A779 | Mouse I779F (14) |
| Ala-A759 | −7 | | Ser-A782 | |
| Gln-A760 | −33 | | Gln-A783 | |
| Cys-A764 | 0 | | Val-A787 | Caenorhabditis elegans |
| | | | | C777Y(15) |
| Val-A765 | −2 | | Val-A788 | |
| Gly-A766 | −1 | | Gly-A789 | |
| Gln-A767 | −34 | N to AMA pos. | Gln-A790 | |
| | | 4 O 3.1 Å | | |
| | | O to AMA pos. | | |
| | | 5 N 3.2 Å | | |
| Gln-A768 | −16 | OE1 to AMA | Gln-A791 | |
| | | pos. 3 O 2.6 Å | | |
| Ser-A769 | −37 | N to AMA pos. | Asn-A792 | Mouse N792D (14) |
| | | 2 O 3.3 Å | | |
| Gly-A772 | −24 | | Gly-A795 | C. elegans G785E (15) |
| Lys-A773 | −4 | | Lys-A796 | |
| Arg-A774 | −2 | | Arg-A797 | |
| Tyr-A804 | −2 | | Tyr-A827 | |
| His-A816 | −13 | | His-A839 | |
| Gly-A819 | −19 | | Gly-A842 | |
| Gly-A820 | −8 | | Gly-A843 | |
| Glu-A822 | −15 | OE2 to AMA | Glu-A845 | |
| | | pos. 2 OD2 | | |
| | | 2.6 Å | | |
| Gly-A823 | −13 | | Gly-A846 | |
| Asp-A826 | −2 | | Asp-A849 | |
| Thr-A1080 | −1 | | Thr-A1103 | |
| Leu-A1081 | −63 | | Leu-A1104 | |
| Lys-A1092 | −37 | | Lys-A1115 | |
| Lys-A1093 | −1 | | Asn-A1116 | |
| Gln-B763 | −16 | | Gln-B718 | |
| Pro-B765 | −11 | | Pro-B720 | |
| Total | −541 | | | |

Δsurface area (Å²) is the change in solvent-exposed surface as calculated with program AREAIMOL, using a standard probe radius of 1.4 Å. Potential hydrogen bonds with a donor-acceptor distance below 3.3 Å were included. Residues that are different between yeast and human are in bold. Mutations are changes in Rpb1 in eukaryotes that are known to affect α-amanitin inhibition. α-Amanitin also seems to make a contact with part of the disordered loop between A1081 and A1092. Unfortunately, only density for ~1 amino acid appears, preventing placement of this loop or even reliable determination of which amino acid in the disordered loop is responsible for this interaction.

Structural derivatives of α-amanitin show the importance of bridge helix interaction for inhibitory activity. The derivative proamanullin, which lacks the hydroxyl group of hydroxyproline 2, involved in hydrogen bonding to bridge helix residue Glu-A822, and which also lacks both hydroxyl groups of 4,5-dihroxyisoleucine 3, is about 20,000-fold less inhibitory than α-amanitin. This effect is caused almost entirely by the alteration of hydroxyproline 2, because alteration of 4,5-dihydroxyisoleucine 3 alone, in the derivative amanullin, reduces inhibition only about 4-fold. Other changes in α-amanitin structure may affect inhibition indirectly, by diminishing the overall affinity for pol II. For example, shortening the side chain of isoleucine-6 of α-amanitin reduces inhibition by about 1,000-fold. This side chain inserts in a hydrophobic pocket of pol II in the cocrystal structure.

Thus three lines of evidence on α-amanitin inhibition, coming from biochemical studies of transcription, from structure-activity relationships, and from cocrystal structure determination, converge on a simple picture. Binding of α-amanitin to pol II permits nucleotide entry to the active site and RNA synthesis but prevents the translocation of DNA and RNA needed to empty the site for the next round of synthesis. The inhibition of translocation is caused by interaction of a-amanitin with the pol II bridge helix, whose movement is required for translocation.

### Example 4

### Complete RNA Polymerase II Complex

For structural studies of complete, 12-subunit pol II, the enzyme was initially isolated from yeast cells grown to stationary phase, where almost all pol II is in the complete form. The resulting crystals were poorly ordered, likely due to the persistence of some core pol II. To overcome the difficulty, we prepared a yeast strain bearing an affinity tag on Rpb4 and isolated the complete enzyme, devoid of core pol II, by affinity chromatography. This homogeneous, complete enzyme preparation formed crystals diffracting to about 4 Å resolution.

### Materials and Methods

Yeast strain CB010 with a Tandem Affinity Purification tag integrated at the carboxy terminus of Rpb4 was grown on YPD medium to late log phase. Yeast cells were resuspended to a density of 0.5 g/ml in 10% glycerol, 50 mM Tris-Cl pH 8.0, 150 mM potassium chloride, 10 mM DTT and 1 mM EDTA. Cells were lysed using a bead beater and clarified lysate was bound to IgG fast flow beads (Amersham Biosciences). The beads were washed with 10 column volumes of 50 mM Hepes pH 7.6, 500 mM ammonium sulfate, 1 mM DTT and 1 mM EDTA, and then with 5 column volumes of 50 mM HEPES pH 7.6, 100 mM potassium chloride, 1 mM DTT and 1 mM EDTA before elution by cleavage with TEV. The eluate was purified on an 8WG16 antibody column and a DEAE HPLC column.

Pol II was concentrated to 10 mg/ml in a microcon with a 100 kDa molecular weight cutoff in 5 mM Tris-Cl pH 7.5, 60 mM ammonium sulfate and 10 mM DTT. Crystals were grown using the hanging drop method against 100 mM ammonium phosphate buffer pH 6.3, 100 mM NaCl, 5 mM dioxane, 1 mM zinc chloride, 5% PEG 6K, and 20-25% PEG 400. Crystals were frozen directly from the mother liquor. Diffraction data was collected at the Advance Light Source beam line 5.0.2 at 0.98 Å. Diffraction data was reduced using the HKL package.

Molecular replacement was carried out with CNS using the fast direct method. The three current pol II models were used as search models. The transcribing complex model (PDB accession code 1I6H) was found to give the best results and all subsequent steps were performed with this model. Rigid body refinement and group B refinement were performed with CNS (final Rcryst=32.5, Rfree=35.7 to 4.1 Å). A difference map calculated using Sigmaa weighted phases revealed a large difference density on the side of the clamp near the back of pol II (FIG. 1). To improve the phases and remove model bias, the Sigmaa weighted phases were used as a starting point for density modification. With only one molecule per asymmetric unit, the calculated solvent content for the complete pol II crystals is greater than 80% (Matthews coefficient of 6.3). Density modification was performed using CNS with a solvent content of 80%. A polyalanine model of the archaeal Rpb4/Rpb7 homologs was placed in a map calculated from the solvent-flattened phases and rigid body refined using CNS. The archaeal homolog model was then modified using 0 to better fit the observed yeast density. A backbone model (alpha carbon atoms only) of the complete 12 subunit pol II and structure factors has been submitted to the PDB (accession code 1 NIK).

The structure of complete, 12-subunit pol II was determined by molecular replacement with that of core pol II (Table 1). All three previous structures, form 1, form 2, and transcribing complex, were used as search models. The transcribing complex structure gave the highest correlation coefficient and lowest initial R-factor. Rigid body refinement with form 2, allowing the clamp to move, resulted in a position of the clamp essentially the same as that in the transcribing complex. We conclude that under the conditions analyzed here, the complete pol II is in the clamp-closed state. This conclusion is in agreement with results of electron microscopy and single particle analysis of complete pol II, which also revealed the enzyme in the clamp-closed state, showing that this conformation was not induced by crystallization.

TABLE 9
Data for complete pol II structure.
Crystallographic Data

| Space Group | C222(1) |
|---|---|
| Unit Cell, Ang | 224.0 by 394.5 by 284.3 |
| Molecules per asymmetric unit | 1 |
| Solvent content, % | 80 |
| Wavelength, Ang | 0.98 |
| Mosaicity, degree | 0.43 |
| Resolution, Ang | 40-4.1 (4.25-4.10) |
| Completeness, % | 98.8 (96.6) |
| Redundancy | 3.5 (3.0) |
| Unique Reflections | 96820 (9357) |
| I/sigI | 5.9 (1.06) |
| Rsym, % | 10.8 (61.4) |

Model Data

| | Residues | Residues | Identity to | Model | Model |
|---|---|---|---|---|---|
| Subunit | In Seq | In Model | Human | Organism | PDB |
| Rpb4 | 221 | 151 | 32% | Methanococcus | 1GO3 |
| | | | | | chain F |
| Rpb7 | 171 | 170 | 43% | jannaschii | 1GO3 |
| | | | | | chain E |

Values in parentheses correspond to the highest resolution shell.
R_{sym} = Σ_{i,h}|I(i, h) − <I(h)>|/Σ_{i,h}|I(i, h)| where <I(h)> is the mean of the I observations of reflection h. R_{sym} was calculated with anomalous pairs merged; no sigma cut-off was applied.

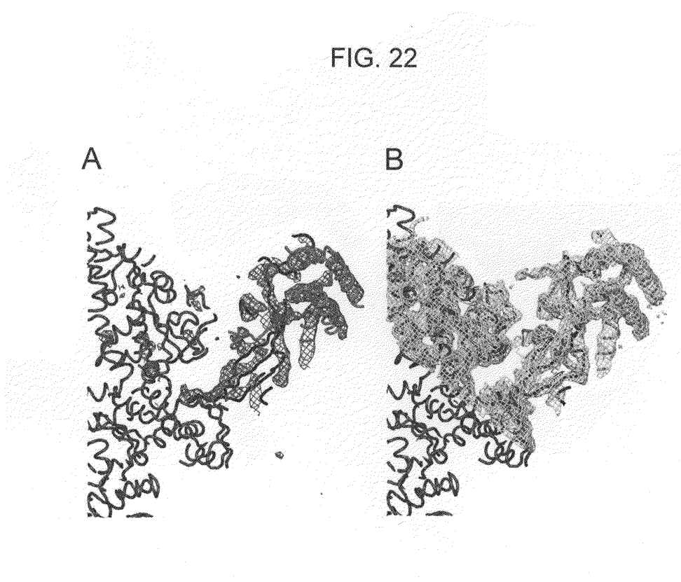

Difference density between the complete and core pol II structures clearly corresponded to the previously reported structure of archaeal Rpb4/Rpb7 (FIG. 22). As the crystals had a high solvent content (Table 9), density modification was performed to improve the map and help remove model bias. A backbone model could be built into the resulting map with the archaeal Rpb4/Rpb7 structure as a guide. The part of the model attributed to Rpb7 was virtually identical to the archaeal structure, in keeping with the sequence conservation between the yeast and archaeal proteins (25% identity, 34% similarity). The remainder of the model, attributed to Rpb4, was very similar to the structure of archaeal Rpb4. There is, however, no significant homology between yeast and archaeal Rpb4 sequences, and most homology between yeast and other eukaryotic Rpb4 sequences is located in the N-terminal 45 and C-terminal 75 residues. We therefore presume that the portion of the Rpb4 structure seen in the map is due to the N- and C-terminal regions; a central, highly charged region of about 70 residues, apparently unique to yeast, is not detected, due to motion or disorder.

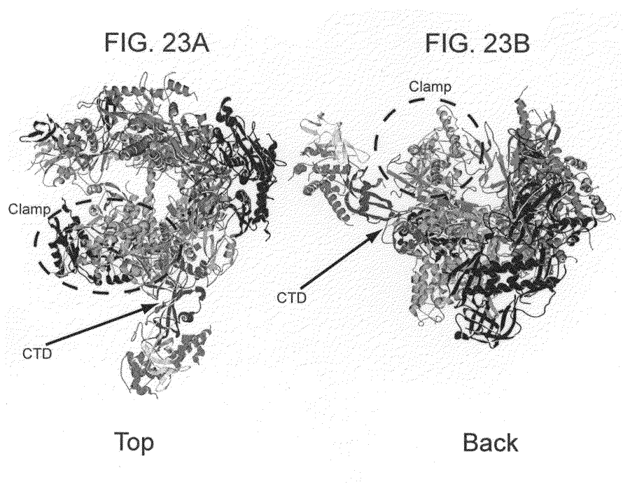

Rpb7 interacts with both Rpb1 and Rpb6 (FIG. 23). Based on alignment with the archaeal structure, a conserved region containing residues 15-20 (numbering scheme from Methanococcus jannaschii) appears to make a hydrophobic interaction with Ala 105 and Pro 106 of Rpb6. In archaeal Rpb7, conserved residues Gly 55, Gly 57, Gly 62 and Gly 64 (M. jannaschii numbering scheme) are located in a loop between two β-strands. In our map, residues corresponding to archeal 55, 57, and 59 appear to be in a β-strand that adds to a β-sheet region of Rpb1 around Val 1443 to Ile 1445, beneath the previously described “RNA exit groove 1”. Residues 62 and 64 are in a loop penetrating the exit groove.

Again using the archaeal structure as a guide, the N-terminal region of Rpb4 makes contact with the N-terminal region of Rpb1 around Ser 8 and Ala 9, located on the surface of the clamp above exit groove 1. Inasmuch as loops in Rpb1 that form the hinge for clamp movement are at the level of the exit groove, contacts of Rpb7 above the groove and Rpb4 below the groove would appear to bracket the clamp, constraining it in the closed state. It seems unlikely that the open conformations of the clamp seen in structures of free core pol II are possible in the presence of the Rpb4/Rpb7 heterodimer. As has been noted, the requirement for the heterodimer for the initiation of transcription, and the effect of the heterodimer upon clamp closure, suggest that promoter DNA binding and initiation occur in the clamp-closed state.

We previously considered the possibility of promoter DNA binding in the clamp-open state, which affords a straight path through the active center cleft for unbent promoter DNA. Binding in the cleft in the clamp-closed state requires bending the DNA to about 90°, and such bending is likely to occur only after interaction with the polymerase and promoter melting. Interaction of straight promoter DNA with pol II in the clamp-closed state may occur as in the structure of the bacterial RNA polymerase holoenzyme-promoter DNA complex, in which the DNA passes above the clamp and adjacent protein “wall”. The DNA presumably descends into the active center region following melting and bending.

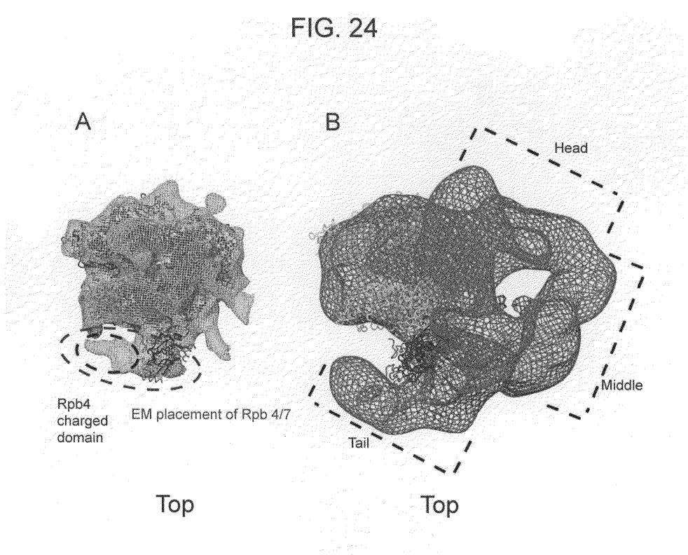

A second implication of the complete pol II structure for transcription concerns the possible involvement of Rpb7 in nucleic acid binding. Rpb7 contains an RNP fold and an OB fold (dark and light blue, respectively, in FIG. 23). The Rpb4/Rpb7 heterodimer was shown to bind single stranded DNA and RNA, and mutation of the OB fold abolished the binding. Previous structure determination of complete pol II by electron microscopy (EM) and single particle analysis placed the heterodimer near RNA exit groove 1, leading to the suggestion that the heterodimer interacts with RNA emanating from the groove. The location of the heterodimer in the X-ray structure agrees well with that determined by EM (FIG. 24A), although the orientation of the heterodimer differs from that previously proposed on the basis of the EM map. It is also consistent with results of immunoelectron microscopy on pol I, which led to the suggestion of heterodimer interaction with the “linker” domain near the C-terminus of Rpb1 (see below). The volume occupied by the heterodimer in the EM map is sufficient to include not only the region of the heterodimer revealed in the X-ray structure, but also the central, charged domain of Rpb4 not seen in the X-ray map (FIG. 24A). Indeed a previous difference electron density map between EM structures of complete and core pol II may have been due entirely to the charged domain.

Details of the heterodimer in the X-ray structure further encourage speculation regarding RNA binding. The surface of the triple-stranded β-sheet of the RNP fold, involved in RNA-binding in other examples of the fold, faces RNA exit groove 1. As already mentioned, a loop containing residues 62 and 64, also involved in RNA-binding in other instances, actually penetrates the groove. The question arises whether the RNP fold of Rpb7 has an affinity for RNA, since mutation of the OB fold abolished RNA binding in vitro. Binding was measured by gel electrophoretic mobility shift analysis, and an affinity constant of micromolar or less, which could significantly affect the stability of a transcribing complex, would have not have been detected. It might be imagined that the RNP fold serves to guide the transcript towards the OB fold, which lies about 50 Å from the exit of groove 1. A transcript length of 25-30 residues would be required to reach the OB-fold, and both capping of the 5′-end and a transition to a stable transcribing complex occur at about this length.

The location of the Rpb4/Rpb7 heterodimer in the complete enzyme suggests a possible role in the assembly of the transcription initiation complex. The heterodimer is adjacent to the site of TFIIB binding in a pol II-TFIIB cocrystal (difference density attributable to TFIIB in the cocrystal is seen near RNA exit groove 1). Evidence for heterodimer-TFIIB interaction, stabilizing the transcription initiation complex, has come from surface plasmon resonance measurements, showing a greater affinity of a TFIIB-TBP-promoter DNA complex for complete pol II than for the core enzyme. Interaction of the heterodimer with TFIIB is also suggested by studies in the yeast pol III system, where the counterpart of Rpb4, termed C17, has been shown to bind the counterpart of TFIIB, termed Brf1, by two-hybrid and co-immunoprecipitation analyses. The location of the heterodimer in the complete enzyme in the vicinity of the C-terminal repeat domain (CTD) (FIG. 23) may be relevant to another reported interaction as well, that of Rpb4 with Fcp1, a phosphatase specific for the CTD.

Finally, the structure of complete pol II has implications for the mechanism of regulation by the multiprotein Mediator complex. Seven additional residues of Rpb1 could be traced in the complete structure beyond the N-terminus seen in the core pol II structure. These additional residues, which appear to interact with Rpb7, form part of the linker between the CTD and the body of pol II (FIG. 23). The CTD is required for the binding of Mediator to pol II. The structure of a Mediator-pol II complex, determined at 35 Å resolution by electron microscopy and single particle analysis, shows a crescent of Mediator density partly surrounding pol II. A gap between a “tail” region of the Mediator and the body of pol II, near the junction of the tail “middle” regions, corresponds to the location of the Rpb4/Rpb7 heterodimer in the X-ray structure (FIG. 24B), raising the possibility of direct Mediator-heterodimer interaction. There is genetic evidence for the involvement of both the heterodimer and Mediator in transcription control: deletion of Rpb4 impairs the activating effect of Gal4 and other yeast regulatory proteins; and deletions of Mediator tail proteins have similar consequences.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1-16. (canceled)

17. A computer-assisted method for identifying potential modulators of eukaryotic transcription, using a programmed computer comprising a processor, a data storage system, an input device, and an output device, comprising the steps of:
(a) inputting into the programmed computer through said input device data comprising three-dimensional coordinates of S. cerevisiae RNA polymerase II enzyme at a resolution equal to or better than 2.8 Angstroms, thereby generating a criteria data set at a resolution equal to or better than 2.8 Angstroms as provided by the structural coordinates of Protein Data Bank Identification Numbers 1I3Q, 1I50, 1I6H and INIK;
(b) comparing, using said processor, said criteria data set to a computer database of chemical structures stored in said computer data storage system;
(c) selecting from said database, using computer methods, chemical structures having a portion that is structurally similar to said criteria data set;
(d) outputting to said output device the selected chemical structures having a portion similar to said criteria data set.

18-19. (canceled)

20. The method of claim 17, wherein said RNA polymerase II is bound to an agent.

21. The method of claim 20, wherein said agent is an inhibitor.

22. A computer-assisted method for identifying potential modulators of eukaryotic transcription, using a programmed computer comprising a processor, a data storage system, an input device, and an output device, comprising the steps of:
(a) inputting into the programmed computer through said input device data comprising three-dimensional coordinates of S. cerevisiae RNA Polymerase II enzyme bound to α-amanitin at a resolution equal to or better than 2.8 Angstroms, thereby generating a criteria data set as provided by the structural coordinates of Protein Data Bank Identification Numbers 1I3Q, 1I50, 1I6H and INIK;
(b) comparing, using said processor, said criteria data set to a computer database of chemical structures stored in said computer data storage system;
(c) selecting from said database, using computer methods, chemical structures having a portion that is structurally similar to said criteria data set;
(d) outputting to said output device the selected chemical structures having a portion similar to said criteria data set.

23. The method of claim 17, wherein said RNA polymerase II is a genetically modified variant of a naturally occurring enzyme.

24. A computer-assisted method for identifying potential modulators of eukaryotic transcription, using a programmed computer comprising a processor, a data storage system, an input device, and an output device, comprising the steps of:
(a) inputting into the programmed computer through said input device data comprising three-dimensional coordinates of a subset of the atoms of S. cerevisiae RNA polymerase II enzyme at a resolution equal to or better than 2.8 Angstroms, thereby generating a criteria data set as provided by the structural coordinates of Protein Data Bank Identification Numbers 1I3Q, 1I50, 1I6H and INIK,
wherein said subset of atoms comprises a structural element selected from the group consisting of rudder, clamp core, clamp head, active site, pore 1, cleft, funnel, and bridge wherein the structural elements comprise the sequence elements as depicted in FIGS. 2A-2C.
(b) comparing, using said processor, said criteria data set to a computer database of chemical structures stored in said computer data storage system;
(c) selecting from said database, using computer methods, chemical structures having a portion that is structurally similar to said criteria data set;
(d) outputting to said output device the selected chemical structures having a portion similar to said criteria data set.

25. (canceled)
